(19) United States
(12) Patent Application Publication
(10) Pub. No.: US 2024/0274292 A1
(43) Pub. Date: Aug. 15, 2024

(54) PREDICTIVE TOOL FOR HEMATOPOIETIC STEM CELL TRANSPLANTATION AND OTHER CELLULAR THERAPY OUTCOMES

(71) Applicant: National Marrow Donor Program, Minneapolis, MN (US)

(72) Inventors: DENIZ AKDEMIR, Ithaca, NY (US); YUNG-TSI BOLON, Roseville, MN (US)

(21) Appl. No.: 18/440,090

(22) Filed: Feb. 13, 2024

Related U.S. Application Data
(60) Provisional application No. 63/484,676, filed on Feb. 13, 2023.

(51) Int. Cl. G16H 50/30; G06N 20/20

(52) U.S. Cl. CPC G16H 50/30; G06N 20/20

(57) ABSTRACT

Methods and systems for risk assessment and trade-off analysis features of a hematopoietic stem cell transplant (HSCT) and cellular therapies decision-making tool are described. In examples, patient outcomes may be predicted and treatment options may be evaluated using a machine learning and data-derived statistical system. Examples described herein are directed to methods for leveraging decision-making support tools for personalized treatment in HSCTs using machine learning modeling and multiple objective optimization.

### CROSS-REFERENCE TO RELATED APPLICATIONS

The present application claims priority from U.S. Provisional Patent Application No. 63/484,676, filed on Feb. 13, 2023, the disclosure of which is hereby incorporated by reference in its entirety.

### BACKGROUND

Cellular therapy in the form of hematopoietic stem cell transplantation has been around for over 50 years and is the only curative treatment option for many diseases, including immunodeficiencies, inherited metabolic disorders, and malignancies. The goal is to restore the body's immune system, either by replacing a patient's immune system with that of a donor or by transplanting autologous or allogenic cells. Hematopoietic stem cell transplant is a complex and expensive procedure that requires considerable care in preparation, execution, and recovery.

The success of hematopoietic stem cell transplantation is dependent on the control of many outcomes and risks such as the prevention of transplantation-related deaths and other complications such as infection, graft-versus-host disease (GVHD), and rejection, while also increasing the effects of the graft versus leukemia and decreasing the risk of relapse. In many cases, these competing objectives are not easily reconciled; a patient may be treated for the primary disease but at the cost of developing secondary complications, such as GVHD. Similarly, an individual may be treated for a secondary complication, but at the cost of a lower quality of life. Other cellular therapies have emerged alongside hematopoietic stem cell transplantation to treat patients.

Personalized medicine, medicine and medical decision-making that considers the unique combination of factors that represent a patient's risks, is an important trend that is gaining momentum in the medical community. However, the growing importance of personalized medicine has not been accompanied by a corresponding increase in research and tools. Being able to make informed decisions about the treatment of a patient requires a comprehensive understanding of the patient's disease, the treatment options, and the potential outcomes of each treatment option.

### SUMMARY

In accordance with examples of the present disclosure, systems and methods for risk assessment and trade-off analysis features of a cellular therapy decision-making tool are described. In examples, patient outcomes may be predicted and treatment options may be evaluated using a machine learning system. Examples described herein are directed to methods for leveraging decision-making support tools for personalized treatment in HSCTs using machine learning modeling and multiple objective optimization.

In a first aspect, a method of evaluating outcomes of a hematopoietic stem cell transplant (HSCT) and other cellular therapies for a patient is provided. The method includes receiving a set of patient characteristics, defining one or more treatment options, and generating data based on the set of patient characteristics and one or more treatment options, including at least one actionable variable. The method further includes determining a risk score associated with a treatment generated using outcomes derived or machine-learned from historical data, defining one or more of those outcomes, and evaluating an impact on the outcome associated with at least one actionable variable. The method includes characterizing the impact of at least one actionable variable on the outcome over a range of values associated with variables to produce a set of pathways based on one or more patient treatment options, identifying and ranking the best options, and generating a visualization of different options.

In a second aspect, a system for evaluating one or more outcomes of a hematopoietic stem cell transplant (HSCT) and other cellular therapies for a patient is provided. The system includes a processor and a non-transitory memory in communication with the processor and storing instructions. The instructions, when executed, cause the processor to generate a risk assessor and a trade-off analyzer. The risk assessor includes one or more machine learning models, each of the one or more machine learning models associated with an outcome and configured to generate a risk score associated with the outcome based on a patient dataset, wherein the patient dataset is associated with a set of treatment options and includes at least one actionable variable. The trade-off analyzer includes a multi-objective optimization framework, wherein the trade-off analyzer uses the multi-object optimization framework to: characterize an impact on the risk score associated with the actionable variable across a range of values for the actionable variable; and identify a non-dominated (Pareto optimal) treatment options based on the set of treatment options, the range of values, and the impact.

A variety of additional inventive aspects will be set forth in the description that follows. The inventive aspects can relate to individual features and to combinations of features. It is to be understood that both the forgoing general description and the following detailed description are exemplary and explanatory only and are not restrictive of the broad inventive concepts upon which the embodiments disclosed herein are based.

### BRIEF DESCRIPTION OF THE DRAWINGS

The accompanying drawings, which are incorporated in and constitute a part of the description, illustrate several aspects of the present disclosure. A brief description of the drawings is as follows:

FIG. 1 illustrates an example operational environment in which aspects of the present disclosure may be implemented.

FIG. 2 illustrates an example computing system with which aspects of the present disclosure may be implemented.

FIG. 3A illustrates a block diagram of an example hematopoietic stem cell transplant (HSCT) decision support system.

FIG. 3B is a flowchart of an example HSCT explorer system.

FIG. 3C is a flowchart of an example process of data preparation.

FIG. 3D is a flowchart of an example process of model training.

FIG. 4 illustrates a flowchart of an example method for performing trade-off analysis.

FIG. 5 illustrates an example of a visualization of variable importance scores.

FIG. 6 illustrates an example of a visualization of dependence analysis for the conditioning intensity variable on transplant outcomes.

FIG. 7 illustrates an example visualizing the effects of several predictor variables on the HSCT two outcomes at a time.

FIG. 8 illustrates an example of trade-off plots for multiple patients.

FIG. 9 illustrates another example output of a trade-off analysis.

FIG. 10 illustrates an example user interface for the HSCT decision assistance tool.

FIG. 11 illustrates a server application flowchart depicting an example operational core driving the user experience and backend functionality.

FIG. 12A is an example graphical user interface (GUI) depicting a predictions user interface (UI) of FIG. 11.

FIG. 12B another example of the GUI of predictions UI identifying inputs and explanation output.

FIG. 12C another example of the GUI of predictions UI identifying outcome selection options.

FIG. 12D is another example of the GUI of predictions UI demonstrating a probability table and plot.

FIG. 12E is another example of the GUI of predictions UI demonstrating a Shapley plot.

FIG. 12F is another example of the GUI of predictions UI demonstrating predictions responsive to changing inputs.

FIG. 12G is another example of the GUI of predictions UI demonstrating prediction outputs for a relapse outcome.

FIG. 12H is another example of the GUI of predictions UI demonstrating inputs and predictive outputs for compare options.

FIG. 12I is another example of the GUI of predictions UI demonstrating the GUI appearance prior to option selection.

FIG. 12J is another example of the GUI of predictions UI demonstrating the GUI appearance with one option selected.

FIG. 12K is another example of the GUI of predictions UI demonstrating the GUI appearance comparing two selected options.

FIG. 12L is another example of the GUI of predictions UI demonstrating the GUI appearance comparing three selected options.

FIG. 12M is another example of the GUI of predictions UI demonstrating operations associated with comparison table output.

FIG. 12N is another example of the GUI of predictions UI demonstrating operations with comparison graph output.

FIG. 13A is an example GUI depicting a trade-off UI of FIG. 11.

FIG. 13B illustrates additional details of the visual output of the trade-off GUI of FIG. 13A.

FIG. 13C illustrates other additional details of the visual output of trade-off GUI of FIG. 13A.

FIG. 13D illustrates other additional details of the visual output of trade-off GUI of FIG. 13A.

FIG. 13E is another example of the GUI of trade-off UI demonstrating inputs and outputs of the UI.

FIG. 13F is another example of the GUI of trade-off UI demonstrating additional details of GUI inputs.

FIG. 13G is another example of the GUI of trade-off UI demonstrating variations in inputs based on selections made.

FIG. 13H is another example of the GUI of trade-off UI demonstrating an SOM mapping presentation of an outcomes view of results.

FIG. 13I is another example of the GUI of trade-off UI demonstrating a table presentation of an outcomes view of results.

FIG. 13J is another example of the GUI of trade-off UI demonstrating a predictor variables view of results with a predictor selected.

FIG. 13K is another example of the GUI of trade-off UI demonstrating a three-dimensional plot of risk scores presentation of an outcomes view of results.

FIG. 13L is another example of the GUI of trade-off UI demonstrating inputs for SOM mapping.

FIG. 14A is an example GUI depicting a Shapley UI of FIG. 11.

FIG. 14B is another example of the GUI of Shapley UI demonstrating inputs and outputs of the GUI.

FIG. 14C is another example of the GUI of Shapley UI demonstrating further detail of the predictor variable selection.

FIG. 14D is another example of the GUI of Shapley UI demonstrating further detail of the Shapely value plot.

FIG. 14E is another example of the GUI of Shapley UI demonstrating another example plot with different inputs.

FIG. 14F is another example of the GUI of Shapley UI demonstrating output changes from the example of FIG. 14E with different inputs.

FIG. 15 is an example GUI depicting a saved results UI of FIG. 11.

FIG. 16A is an example GUI depicting a details UI of FIG. 11.

FIG. 16B is another example of the GUI of details UI demonstrating an usage information page.

FIG. 16C is another example of the GUI of Shapley UI demonstrating a model details view of an overall survival outcome model.

FIG. 16D is another example of the GUI of Shapley UI demonstrating the model details view of an event-free survival outcome model.

FIG. 16E is another example of the GUI of Shapley UI demonstrating the model details view of a relapse outcome model.

FIG. 16F is another example of the GUI of Shapley UI demonstrating the model details view of a rejection outcome model.

FIG. 16G is another example of the GUI of Shapley UI demonstrating the model details view of a graft-versus-host disease outcome model.

FIG. 16H is another example of the GUI of Shapley UI demonstrating a predictor variables view.

FIG. 16I is another example of the GUI of Shapley UI demonstrating a summary view of predictor variables.

FIG. 16J is another example of the GUI of Shapley UI demonstrating a summary view of donor variables.

FIG. 16K is another example of the GUI of Shapley UI demonstrating a summary view of treatment variables.

### DETAILED DESCRIPTION

Reference will now be made in detail to exemplary aspects of the present disclosure that are illustrated in the accompanying drawings. As briefly described above, embodiments of the present invention are directed to methods and systems for evaluating outcomes and comparing treatment options for patients who may need a hematopoietic stem cell transplant (HSCT).

HSCT is a commonly used treatment for various hematological and non-hematological diseases. However, the prognosis of HSCT outcomes can vary greatly depending on several factors such as patient characteristics, disease type, and transplantation regimen. Accurately predicting the prognosis of HSCT and other cellular therapy outcomes and selecting the best treatment options for each patient is crucial for patient care and clinical trial design. The objectives and risks of HSCTs need to be clearly articulated in the decision-making process for effective patient treatment, a problem well-suited to being addressed through the use of data-driven decision support tools. There is presently a scarcity of evidence-based decision support tools to aid in the decision-making process for HSCT and other cellular therapies and, without such tools, the clinical practice remains challenging, time-consuming, complex, and requires a high level of expertise.

Disclosed herein is a set of machine learning based decision support tools for HSCT and cellular therapies in general, aimed at assisting practitioners in making informed decisions about the treatment of blood diseases. The present disclosure is directed to the use of trade-off analysis, a multi-objective optimization framework, and various visualizations to predict the risks related to different HSCT treatment options and aid in decision making, though other features and tools are contemplated and may be incorporated into a common platform which represents a transformative step towards data-driven medicine.

In example aspects presented herein, systems and methods for risk assessment and trade-off analysis features of a cellular therapy decision-making tool are described. In examples, patient outcomes may be predicted and treatment options may be evaluated using a machine learning system. Examples described herein are directed to methods for leveraging decision-making support tools for personalized treatment in HSCTs using machine learning modeling and multiple objective optimization. Multiobjective optimization provides a robust framework for balancing different patient outcomes, thereby aiding in making informed decisions that optimize patient care while considering the unique risks and benefits for each individual.

Examples presented herein use data-driven statistical methods and machine-learning systems for prognostic prediction of the outcomes from HSCTs and other cellular therapies, to identify relationships between actionable factors and HSCT outcomes, and to introduce trade-off analysis methods to aid in the complex decision-making tasks for the improvement of the overall outcome of HCTs.

Patient outcomes may be evaluated using outcome-specific machine learning models to predict outcomes based on patient characteristics. Treatment options may be evaluated using a trade-off analysis and a multi-objective framework to analyze predicted patient outcomes and identify optimal treatment options for a particular patient based on patient characteristics, outcome predictions, and treatment options available.

### I. Operational Environment for Diagnostic Toolsets

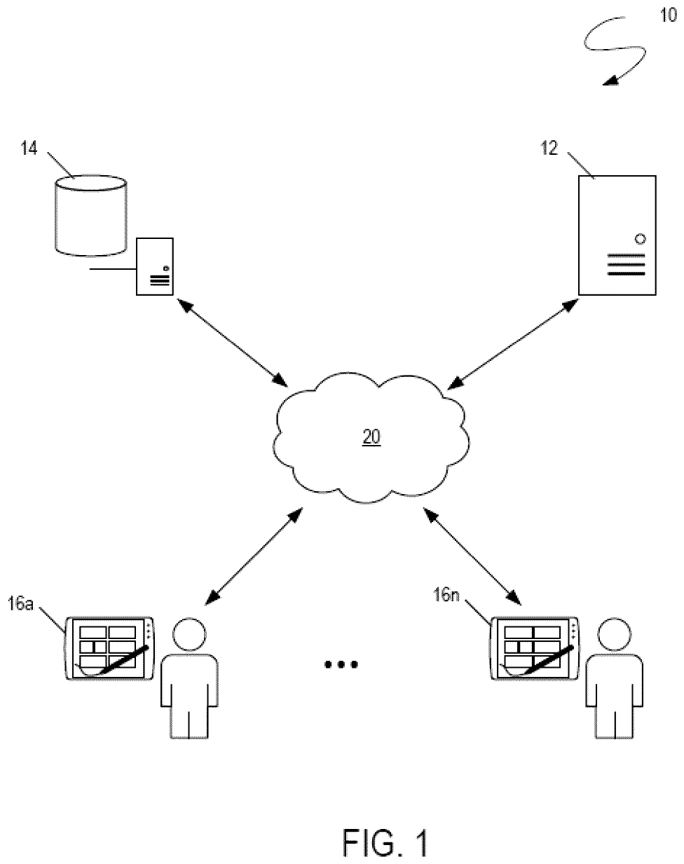

FIG. 1 illustrates an example network environment 10 in which aspects of the present disclosure may be implemented. In particular, the environment 10 may be used to provide one or more diagnostic toolsets to clinicians for purposes of assisting with determining optimal treatment programs for patients. The diagnostic toolsets can include the machine learning based decision support tools for HSCT as discussed below.

In the example shown, a diagnostic analysis server 12 may be communicatively connected to a treatment outcome repository server 14 via a network 20, such as the Internet. The diagnostic analysis server 12 may host one or more analysis programs, such as machine learning models and/or multi factor optimization analysis programs, which may use treatment outcome data from the treatment outcome repository server 14 for purposes of analysis and prediction. One or more clinicians, via clinician computing devices 16a-n (collectively referred to as clinicians 16 herein, for simplicity), may access one or more diagnostic tool sets provided by the diagnostic analysis server 12, for example to determine an optimal course of treatment for a particular patient.

The diagnostic analysis server 12 may host a variety of types of diagnostic tools. An example diagnostic tool may implement the risk assessor 202 and trade-off analyzer 214 of the HSCT decision support system 200 described below. Other types of tools may be implemented as well.

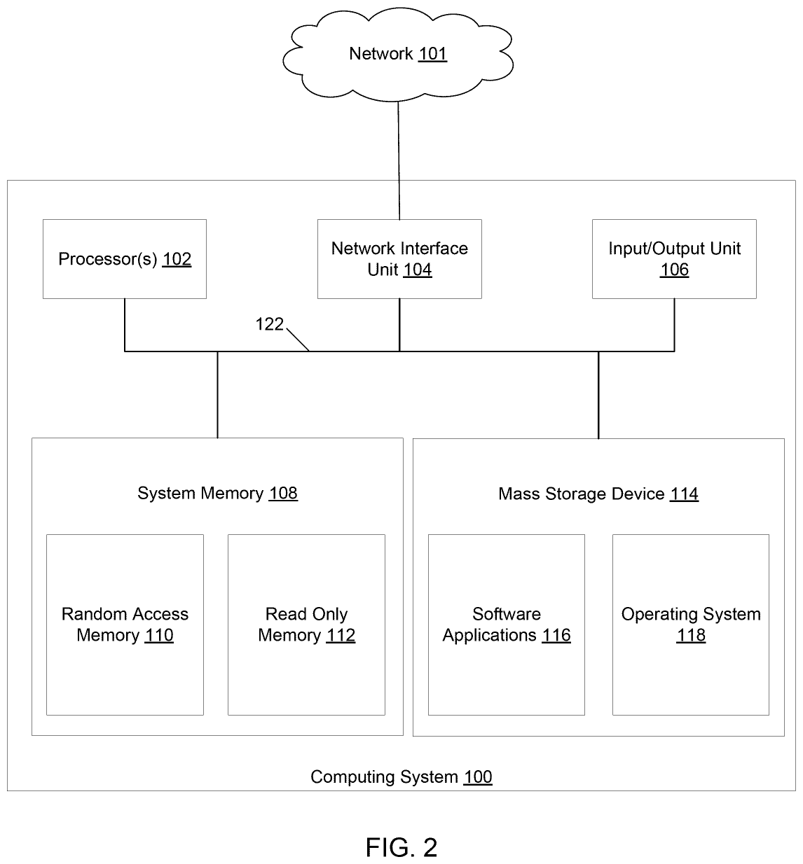

FIG. 2 illustrates an example block diagram of a virtual or physical computing system 100. One or more aspects of the computing system 100 can be used to implement the systems and methods for risk assessment and trade-off analysis described herein. In particular, the computing system 100 may be used to implement the diagnostic analysis server 12, the treatment outcome repository server 14, or the one or more of the clinician computing devices 16a-n.

In the embodiment shown, the computing system 100 includes one or more processors 102, a system memory 108, and a system bus 122 that couples the system memory 108 to the one or more processors 102. The system memory 108 includes RAM (Random Access Memory) 110 and ROM (Read-Only Memory) 112. A basic input/output system that contains the basic routines that help to transfer information between elements within the computing system 100, such as during startup, is stored in the ROM 112. The computing system 100 further includes a mass storage device 114. The mass storage device 114 is able to store software instructions and data. The one or more processors 102 can be one or more central processing units or other processors.

The mass storage device 114 is connected to the one or more processors 102 through a mass storage controller (not shown) connected to the system bus 122. The mass storage device 114 and its associated computer-readable data storage media provide nonvolatile, non-transitory storage for the computing system 100. Although the description of computer-readable data storage media contained herein refers to a mass storage device, such as a hard disk or solid state disk, it should be appreciated by those skilled in the art that computer-readable data storage media can be any available non-transitory, physical device or article of manufacture from which the central display station can read data and/or instructions.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, DVD (Digital Versatile Discs), other optical storage media, magnetic cassettes, magnetic tape, magnetic disk storage or other magnetic storage devices, or any other medium which can be used to store the desired information and which can be accessed by the computing system 100.

According to various embodiments of the invention, the computing system 100 may operate in a networked environment using logical connections to remote network devices through the network 101. The network 101 may correspond to network 20 of FIG. 1, and is a computer network, such as an enterprise intranet and/or the Internet. The network 101 can include a LAN, a Wide Area Network (WAN), the Internet, wireless transmission mediums, wired transmission mediums, other networks, and combinations thereof. The computing system 100 may connect to the network 101 through a network interface unit 104 connected to the system bus 122. It should be appreciated that the network interface unit 104 may also be utilized to connect to other types of networks and remote computing systems. The computing system 100 also includes an input/output controller 106 for receiving and processing input from a number of other devices, including a touch user interface display screen, or another type of input device. Similarly, the input/output controller 106 may provide output to a touch user interface display screen or other type of output device.

As mentioned briefly above, the mass storage device 114 and the RAM 110 of the computing system 100 can store software instructions and data. The software instructions include an operating system 118 suitable for controlling the operation of the computing system 100. The mass storage device 114 and/or the RAM 110 also store software instructions, that when executed by the one or more processors 102, cause one or more of the systems, devices, or components described herein to provide functionality described herein. For example, the mass storage device 114 and/or the RAM 110 can store software instructions that, when executed by the one or more processors 102, cause the computing system 100 to receive and execute managing network access control and build system processes.

Referring to the figures and examples presented herein generally, the disclosed environment provides a physical environment with which aspects of the outcome prediction and trade-off analysis systems can be implemented.

### II. HSCT Decision Support Tool and Methods

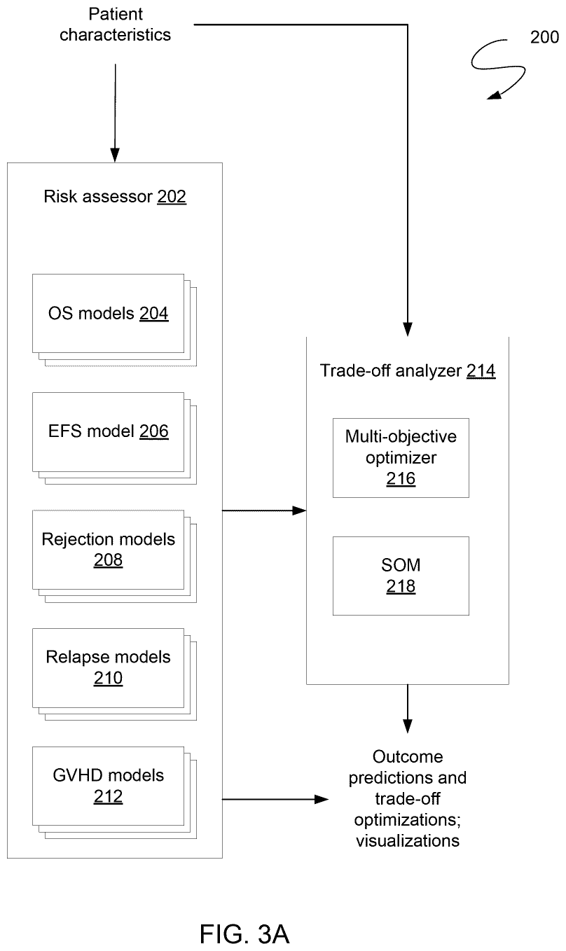

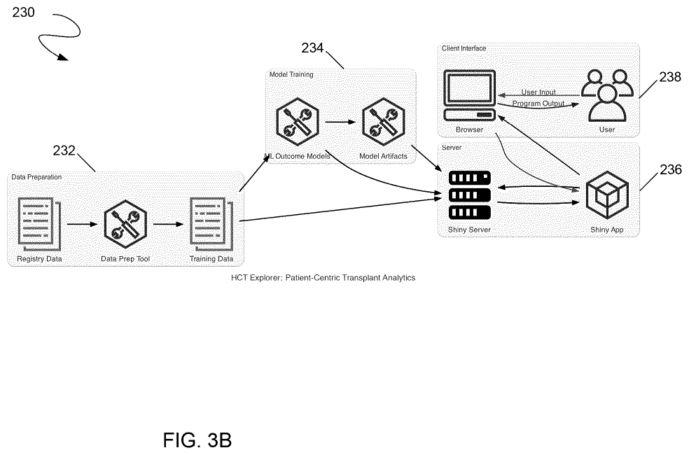

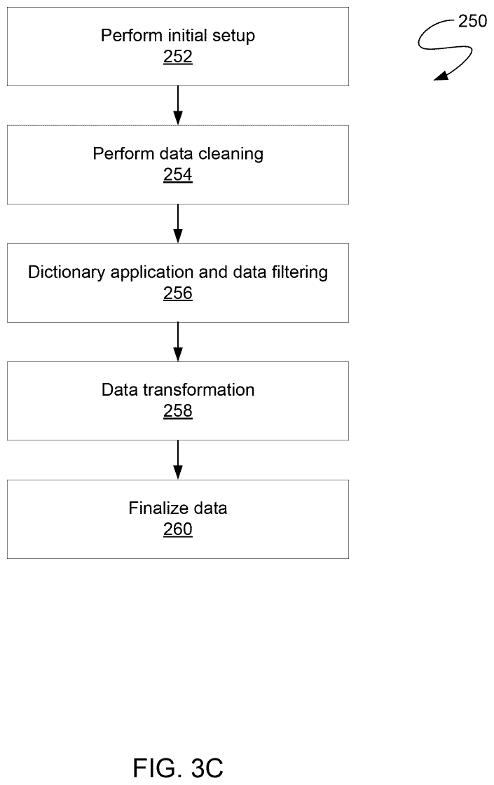

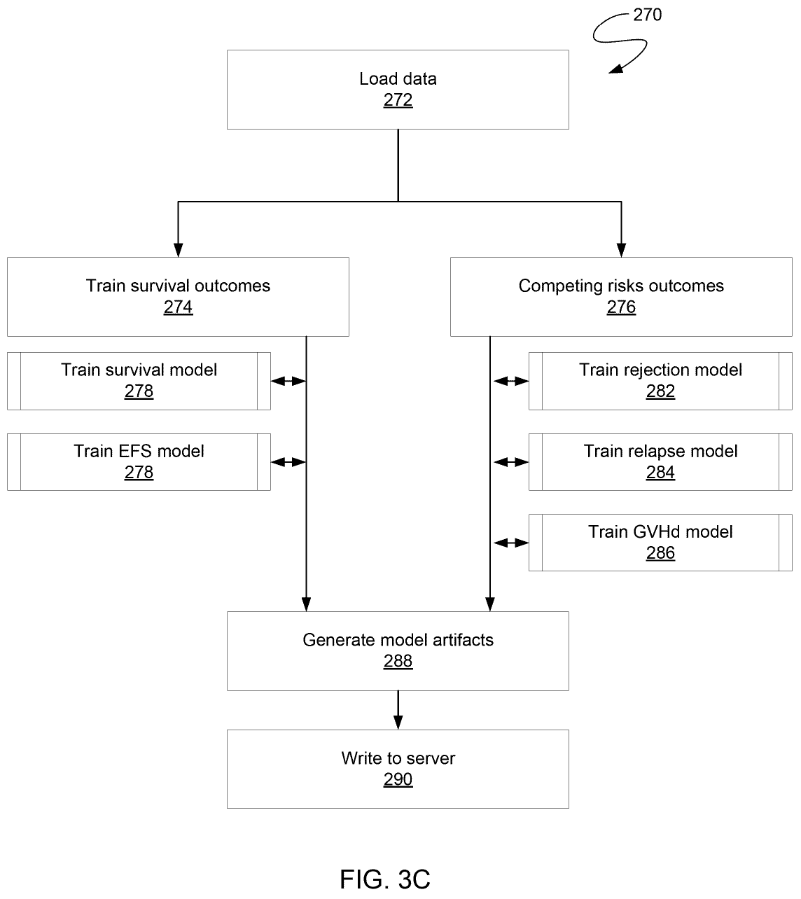

Referring now to FIG. 3A, a block diagram of an example HSCT decision support system 200 is shown. HSCT decision support system 200 may include a risk assessor 202, including one or more outcome models 204-212, and a trade-off analyzer 214 including a multi-objective optimization framework 216 and a self-organizing map (SOM) 218. The present disclosure is directed to particular features of the HSCT decision support system 200, including risk assessor 202 and trade-off analyzer 214, but HSCT decision support system 200 is contemplated to include other features and capabilities not discussed in detail herein.

In examples, HSCT decision support system 200 may be a component of computing system 100. In examples, HSCT decision support system 200 may be a program executed on or by computing system 100, such as a web-based program executed via network 101. HSCT decision support system 200 may represent instructions stored locally, such as in mass storage device 114, or remotely, such as on a cloud-based platform accessed via network 101.

Risk assessor 202 quantifies a patient's risks in terms of a likelihood of one or more defined outcomes. In examples, risk assessor 202 may be configured to perform time-to-event analysis. Time-to-event analysis refers to a set of statistical methods for analyzing data relating to the duration of one or more events. Examples of time-to-event outcomes include the occurrence of a disease, or death. Time-to-event outcomes can be measured on a continuous scale using a fixed time interval, such as daily, monthly, etc.

Risk assessor 202 includes one or more machine learning models configured to quantify a patient's likelihood of a defined outcome. Non-limiting examples of outcomes that may be predicted are overall survival (OS), event-free survival (EFS), rejection of the transplant, relapse, and graft-versus-host disease (GVHD). As discussed herein, OS refers to the time from transplantation until death. EFS refers to the time from transplant until death or until any one of relapse, rejection, or GVHD.

Example risk assessor 202 includes OS models 204, EFS models 206, rejection models 208, relapse models 210, and GVHD model 212. A set of one or more predictive models is trained to determine a risk for each outcome, e.g., a time to the particular outcome. Each of outcomes models 204-212 may represent a single machine learning model or a set of two or more machine learning models. A separate model may be trained to determine a risk for each defined outcome. In examples, multiple models, or a model set, may be trained to determine a risk for each defined outcome.

Machine learning for time-to-event modeling refers to models configured to predict, based on a set of predictor variables, a probability of the occurrence of a given event within a predetermined time period. Time-to-event data may be censored or truncated. Truncation and censoring mechanisms may be independent of the time-to-event variable.

Modeling approached may be adjusted to accommodate censoring or truncation of time-to-event data and may differ from conventional machine learning regression or classification tasks. In embodiments, OS and EFS outcomes may be analyzed as survival outcomes, such as right-censored survival outcomes. Rejection, relapse, and GVHD outcomes may be analyzed as competing risk events, where for each of these outcomes the competing risk is defined as death without this outcome. Competing risk outcomes may be censored or truncated, such as being analyzed as right-censored competing risk outcomes.

Many models with different properties may be appropriate to use with survival or competing risk outcomes. Machine learning models for time-to-event analysis may be categorized under broad categories, such as parametric or non-parametric based on whether the event times are assumed to follow a known probability distribution, or not or additive or non-additive based on whether the model assumes the overall effect of the terms in a model is the sum of the effects of individual terms in the model. Additive models generally require explicit use of predictor variables that are designed to capture interactions, while non-additive models can be used to implicitly capture interaction effects. Further, any of these models, parametric or non-parametric, additive or non-additive, can be approached with either classical or Bayesian inference.

Certain non-limiting examples of machine learning and other data-driven statistical models appropriate for analysis of survival data for outcomes such as OS and EFS are: a Cox proportional hazards (CPH) model; a Random survival forest (RSF) model using either all of the predictor variables or fitting a first model and using a number of the top ranked most important variables selected from the first model; an gradient boosted machine model (GBM) or an extreme Gradient Boosting model (XGBoost) iteratively combining weak learners into a single strong learner where each consecutive learner is trained to improve the predictions of outcomes that have larger errors; a rule-based model fitting sparse linear models and automatically detecting interaction effects in the form of binary decision rules; a parametric survival regression model using exponential or Weibull distributions; a Bayesian Additive Regression Trees (BART) model for survival outcomes which does not assume that relationships between covariates and the outcome will follow a linear form; or an ensemble or combination survival model. Variable selection for survival models may be by step-wise variable selection using, for example, Akaike's information criterion (AIC) or an automatic selection of variables with or without regularization of effects based on an elastic-net.

Certain non-limiting examples of machine learning and other data-driven statistical models appropriate for the competing risks data for outcomes such as rejection, relapse, and GVHD are: a Fine Gray Regression; a multi-state competing risks model; a random forest for competing risks fit using either all of the predictor variables or fit using a number (e.g., 10, 20, 30, etc.) most important variables based on a selection from a fit of a first model; a rule-based model; a BART model for competing risks; an ensemble or combination competing risks model. In embodiments, a CPH model with variable selection using AIC and considering death outcomes as censored may be used particularly for evaluating the effects of predictor variables. Variable selection for competing risks models may be made by stepwise variable selection using AIC or automatic variable selection of variables with or without regularization of the effects based on an elastic-net.

In embodiments, model selection may include a model performance evaluation. A variety of models may be tested to determine which yields the best performance based on a set of training data. Factors considered in evaluating model performance may include choice of predictor variables, sample size, nature of relationships among predictor variables, and effects of predictor variable on the transplant outcomes. Model selection may further account for availability of models and resources, ease of implementation, suitability for integration with pre-existing systems, computation efficiency, etc. Those of skill in the art will understand that other models will be suitable for the functions and configurations described herein and that model selection can be adjusted without making a significant effect on the subject matter of the present disclosure.

Models may be selected with consideration to explainable artificial intelligence (AI). Explainable AI is an approach that seeks to explain the predictions made by the machine learning models and may be used to provide insights into why a model made a particular prediction. Integration of explainable AI can help clinicians make more informed decisions about the treatment of HSCT patients, can help to improve the accuracy of the models over time, can help to increase trust in machine learning, and can help to identify potential areas of bias or errors in the decision-making process.

Explainable AI for survival analysis is a set of techniques used to explain the results of survival analysis models. These techniques can be used to explain how the model is making its predictions, and why it is making them. Some non-limiting examples of these techniques are feature importance, partial dependence plots, Shapely Additive Explanations (SHAP) values, local interpretable model-agnostic explanations (LIME), and global interpretable model-agnostic explanations (GIME).

Feature importance is used to identify which features have the most influence on the model's predictions and can help identify which variables are most important for predicting survival outcomes. Partial dependence plots examine the influence of a single feature on the model's predictions and can be used to understand how changes in that single feature's values affect the model's predictions.

SHAP values are used to explain the contributions of each feature to the model's predictions. SHAP values can be used to compare the relative importance of different features in the model. LIME is used to explain the predictions of the model in a localized manner. LIME can be used to explain why the model makes a particular prediction for an individual case. GIME is used to explain the predictions of the model globally. GIME can be used to explain the overall effect of different features on the model's predictions.

Explainable AI features can be integrated with individual models of the HSCT decisions support system, or may be integrated with the overall system and apply to the use of some of all of the machine learning models in the system. In embodiments, common explainable AI features may integrate with features discussed herein, including risk assessor 202 and trade-off analyzer 214, and other features of HSCT decisions-support system 200.

Models for each of the defined outcomes may be trained simultaneously, with the exception ensemble models requiring input from other trained models. Models may be trained using data from transplant records with defined variables. For example, the Center for International Bone Marrow Transplant Research maintains a database of patients undergoing HSCT. At the time of filing, this database contains over 30,000 unique transplant records with over 100 variables. Such a database provides a robust source of training and validation data for the machine leaning systems and method of the present disclosure.

Examples presented herein may recite particular variables or numbers of variables, but those of skill in the art will understand that other variables can be identified or incorporated while adhering to the principles disclosed herein.

Some variables may be characterized as predictor variables. Predictor variables are independent variables that influence dependent variables. Predictor variables can be fixed or free. Free predictor variables may be actionable variables, or variables that can be adjusted to influence outcomes. Some non-limiting examples of possible predictor variables include: donor age at the time of HSCT, recipient age at the time of HSCT, medications or other treatments the recipient is receiving in preparation for or at the time of HSCT, matching between the donor and the recipient, recipient cytomegalovirus (CMV) status, disease or indication for transplant, recipient disease status, graft source, GVHD prophylaxis, history of fungal infection, history of mechanical ventilation use; median household income for recipient's zipcode, recipients Karnofsky performance score, recipient's race, whether the donor and the recipient are related, recipient sex, and the interval from diagnosis to transplant.

Examples of medications or treatments a HSCT recipient may be receiving as part of the therapy include antithymocyte globulin, Alemtuzumab, reduced intensity and myeloablative conditioning, including total body irradiation (TBI) dose before HSCT, and various GVHD prophylaxis regimens. The intensity of conditioning, e.g., radiation therapy or chemotherapy, the recipient is receiving in preparation for or at the time of HSCT may be a predictor variable. The intensity of the conditioning may be based on the type and status of HSCT, the patient's age, and the patient's health status.

Donor and recipient matching looks at match or mismatch of different factors between donor and recipient. Matches may be simple match/no match metrics or graded scales. Matching factors may be represented by a single variable, e.g., a match/no match variable, or one or more variables, e.g., a match score for the donor and a match score for the recipient. Some examples of matches evaluated between donors and recipients in HSCTs are cytomegalovirus (CMV) serostatus, whether the donor and the recipient are the same sex, and human leukocyte antigens (HLA) match. Different levels of HLA matching may be considered. For example, an HLA match 6 evaluates matching at HLA-A,-B, and-DRB1; HLA match 8 evaluates matches at HLA-A,-B,-C, and-DRB1; and HLA match 10 evaluates matches at HLA-A,-B,-C,-DRB1, and-DQB1. One or more levels of HLA matching may be included as variables.

Due to the overlap between different levels of HLA matching, these variables can be transformed before training or input. For example, by transforming an HLA match 10 variable by subtracting corresponding HLA match 8 variable values. Because the HLA match 10 variable is the number of HLA matches out of 10 HLA loci and HLA match 8 is the number of HLA matches out of 8 HLA loci, the difference between the two variables is the number of HLA matches in HLA-DQB1 region. HLA match 8 can be similarly transformed by subtracting HLA match 6 variable values. The difference between the two variables is the number of HLA matches in HLA-C region. This may make the HLA matching variables less dependent although there may still be a high level of dependence among the final variables due to high linkage disequilibrium among the HLA genes.

Some variables may be related to a particular outcome or disease. Outcome-related variables are dependent variables to indicate a particular patient's outcomes after cellular therapy when known. Examples of outcome-related variables include death status, time to death, event free survival status, time to event-free survival status, GVHD status, time to a first moderate or severe GVHD event, graft failure or rejection status, time to rejection, clinical or hematological relapse status, and time to clinical or hematological relapse.

If a variable is disease-specific, meaningful values for this variable are only possible for a certain set of diseases. This is worth noting for distinguishing between cases where a value is absent due to being unrelated to the disease for that particular data set and when it is truly missing. Cytogenetic scores are an example of a disease specific variable. Cytogenetic score is a measure of the genetic abnormalities in cancer cells, it is based on the number and type of abnormalities that are present in the cancer cells. A separate cytogenetic score may be determined for each kind of cancer or group of cancers, e.g., acute lymphocytic leukemia (ALL), acute myeloid leukemia (AML), chronic myeloid leukemia (CML), myelodysplastic (MDS) syndromes as a group, MDS transformed to AML, presence or absence of the Philadelphia chromosome with ALL, and therapy related AML. Cytogenetic scores may be determined by the system or retrieved from another source. For example, an international standard for scoring cytogenetic scores for Myelodysplastic Syndromes, the International Prognostic Scoring System-Revised, may be used.

In examples, disease-specific cytogenetic scores may be transformed to yield an overall cytogenetic score, such as by fitting a CPH model in the training data with multiple disease specific cytogenetic scores as predictors and the OS outcome as the response variable. The fitted model may then used to predict the risk scores (exponentiated linear score which can be interpreted as the hazard rate) for the whole data This method of feature extraction may be compared to weight of evidence feature extraction. Another example of disease specific variables is a patient's ALL immunophenotype, referring to antigens uses to classify ALL into subtypes.

A body of transplant data records may be retrieved and filtered to yield a working body of data suitable for training and validation of the models. In examples, the transplant data records may be obtained from the treatment outcome repository server 14, as discussed previously. The body of data may be split randomly into two portions. The portions may be unequal, with a larger portion used for training the machine learning models. The training data portion may be used during both variable selection and model fitting.

The remaining portion may be retained for model evaluation. Trained models may be evaluated for fit using validation data. The validation data may generally be additional data reflecting the training data. Validation data may be used to evaluate the generalization performance of the models to unseen observations. Model performance evaluation using the training data may over-predict the models' generalization performance. The estimates of performance from the validation data (which was not used in the training of the model) may better approximate the true performances of the models.

Trained models are used to generate predictions based on the validation data. Predictions may be compared with the observed outcomes to calculate time-dependent concordance index values. The time-dependent concordance index is a measure of how much better the model predicts the data than chance at a certain time. Its value at a given time can be interpreted as the probability of a model correctly predicting the outcome status of an individual at a certain time. Another measure of interest for comparing the accuracy of models is Harrell's c-index. Harrell's c-index measures the probability that the predictions for a random pair of subjects are concordant with their outcomes. The ability of the time-dependent concordance score to quantify performance at different times can be valuable for the evaluation and selection of models.

In examples, the GAM model or an ensemble of a plurality of trained modes may be preferred for survival outcomes, OS and EFS, for the having the greatest accuracy. Survival model in general may be relied upon to have consistent accuracy independent of the time period evaluated. In examples, for competing risks outcomes, the rule-based model or the ensemble of a plurality of the trained models may be preferred. In general, the competing risks models may tend to lose accuracy over time.

Once trained, outcome models 204-212 may be used by risk assessor 202 to predict the survival functions or the cumulative incidence function (CIF) over a fixed time interval. In embodiments incorporating ensemble models, the predictions from these models are used to obtain ensemble predictions for the survival functions or the competing outcomes functions. A risk score may be output for each outcome measuring an amount of time lost in a given time period (e.g., 18 months from HSCT) and may be calculated using estimated functions and numerical integration. These predictions may also form the basis for evaluating the nature of the effects of predictor variables on the HSCT outcomes.

Outcome models 204-212 may also be used to make other inferences, such as plots of survival functions or competing risks functions, median survival time, probability of survival or incidence at a given time point, and hazard functions. These inferences include predictions for new data, estimates for the model parameters, measures of variable importance, and the size and direction of effects for predictor variables. Inferences from models can be based on model-specific methods which are limited to specific model classes (for instance, estimates for the model parameters for a CPH model) or model-agnostic tools which can be used with any machine learning model. Some non-limiting examples of model-agnostic machine learning model interpretation methods include: permutation variable importance, partial dependence plots, accumulated local effects plots, break down plots, and ceteris paribus analysis.

In some examples, a reference data based conditional approach may be preferred over methods such as partial dependence and ceteris paribus analysis, because predictor variables in HSCT data may not independent, and therefore every predictor variable cannot be freely permuted while keeping others constant. A physician or other user may set parameters for a dataset to be used when making outcome predictions or performing trade-off analysis. For example, predictor variables related to recipient factors that may be fixed to match a current patient's characteristics, or donor variables may be limited to a range set according to available donor characteristics.

Predictions output by risk assessor 202 using outcome model 204-212 may also form the basis of trade-off analysis performed by trade-off analyzer 214 using a multi-objective optimization framework 216 and self-organizing map (SOM) analysis 218.

Clinical decisions for HSCTs and other cellular therapies involve multiple, often competing, objectives, including, for example, cure or control of the disease, prevention of complications, prevention of infections, preservation of rejection, prevention of graft versus host disease, etc. Each transplant and treatment strategy can be associated with a unique compromise between many different objectives. Trade-off analyzer 214 provides multi-objective treatment recommendations based on a multi-objective optimization framework by learning optimal compromise solutions, e.g., a Pareto front, via a complete search of different transplant and treatment strategies in terms of several HSCT outcomes. The provided treatment recommendations can be used to help in the medical decision-making process to carry out the best choice for a given patient.

Using multi-objective optimization principles along with machine learning prediction models for predicting the risks associated with different treatment possibilities for a given patient can be used to evaluate the trade-offs of HSCTs and other cellular therapies and provide data and visualizations useful for making individualized treatment decisions.

Multi-objective optimization problems require the simultaneous optimization of several objectives. Although single-objective optimization problems may have a unique optimal solution, multi-objective optimization problems (as a rule) present a multiplicity of compromise solutions, e.g., Pareto optimal solutions. These solutions are referred to as non-dominated solutions, as in they dominate the rest of the solutions, and are defined as a solution where no variable can be further improved without detriment in another variable. Several algorithms are known in the art for finding the non-dominated set in a large set of solutions. Some non-limiting examples of such algorithms are the Kung algorithm or the Jun Du Algorithm.

For visualizing the Pareto optimal solutions, standard scatter plot may be used for low-dimensional problems, while self-organizing maps (SOMs) may be preferred for higher-dimensional (e.g., more than 3 dimensions) problems. A self-organizing map is a neural network trained to produce a topological map. An input layer of the SOM has the points in the set and an output layer has the coordinates of each point on a map. The SOM uses the points in the set to construct a topological map of the set. The mapping produced by SOMs for risks associated with several HSCT outcomes can be plotted from different perspectives to study the relationship of the outcomes to the predictor variables. A mapping of the Pareto optimal treatment options with respect to several predicted risks can be used to study the trade-offs among the outcomes with respect to the values of the predictor variables.

Referring now to FIG. 3B, a flowchart of an example HSCT explorer system 230 is shown. In embodiments, explorer system 230 uses a decision support system, such as decision support system 200 of FIG. 3A, to provide a predictive analysis framework. As shown in FIG. 3B, explorer system 230 consolidates data preparation element 232, model training elements 234, and result interpretation elements, such as application server 236 which supports a user interface 238.

Data preparation elements 232 encompass the training data itself and preparation tools applied to generate the training data from raw data. Data preparation elements 232 may form a foundation of the HSCT explorer system, emphasizing the importance of rigorous data handling in ensuring accuracy of a predictive model. Raw information is refined and transformed into a structured format to ensure suitability for application to advanced machine learning techniques. The fidelity of this process ensures that the resulting predictive models stand on solid ground, providing reliable and clinically relevant insights that drive the decision-making process.

FIG. 3C is a flowchart of an example process 250 of data preparation. At operation 252, initial setup is performed, including, for example, importing libraries and loading data. At operation 254, data cleaning is performed, including, for example, data frame conversion and unique data cleaning. At operation 256, one or more dictionaries may be applied to standardize terms and the data may be filtered to meet specific criteria. Operation 256 helps maintain consistency across the dataset. At operation 258, the data may be transformed including, for example, variable renaming. In embodiments, data transformation ensures the data aligns with the requirements of one or more predictive models. Variables may be renamed for clarity and ease of understanding. At operation 260, the data is finalized. In embodiments, finalizing the data is dedicated to fine-tuning the dataset, ensuring it is in the optimal format for model training. The prepared dataset is then saved, marking the completion of the data preparation process.

This approach to data preparation ensures the accuracy and reliability of the predictive models in the HSCT Explorer system 230. Data preparation process 250 ensure a dataset is craft that is both precise and tailored for the training of machine learning models, ultimately ensuring that the HSCT Explorer system 230 can deliver reliable and accurate predictions for patient-centric healthcare solutions.

Model training elements 234 encompass raw model design and training, as well as any model artifacts produced from the training process. For example, the models 204-212 of risk assessor 202 in FIG. 3A represent example artifacts of model training elements 234.

FIG. 3D is a flowchart of an example process 270 of model training.

At operation 272, data is loaded. At operations 274 and 276, model training for survival outcomes and competing risks outcomes is executed. In embodiments, operations 274 and 276 may be executed in sequence or in parallel. Each outcomes operations 274 and 276 includes subprocess of training specific models for the relevant outcomes. For example, survival outcomes training operation 274 includes training a survival model (which may coincide with OS models 204 of FIG. 3A), at operation 278, and an EFS model (which may coincide with EFS models 206 of FIG. 3A), at operation 280. Risk outcomes training operation 276 may include training a rejection model (which may coincide with rejection models 208 of FIG. 3A), at operation 282, a relapse model (which may coincide with relapse models 210 of FIG. 3A), at operation 284, and a GVHD model (which may coincide with GVHD models 212 of FIG. 3A), at operation 286. At operation 288, model artifacts are generated using the training of the models at operations 278-286. At operation 290, the model artifacts are written to the server, such as application server 236 in FIG. 3B, for subsequent use.

In embodiments, application server 236 is a Shiny application server and enables clinical decision support through interactive web-based user engagement provided by user interface 238. Application server 236 may correspond to trade-off analyzer 214 of FIG. 3A, and encapsulates a novel method of forecasting transplant outcomes, leveraging multi objective analysis and SOM clustering to offer individualized treatment insights and strategic decision-making support for clinicians via user interface 238. By blending the advanced data analytics provided by application server 236, with a user-friendly interface, the system and processes disclosed herein enables clinicians to make well-informed decisions by analyzing individual patient data, treatment options, and their outcomes. The predictive tool offers a novel approach to forecasting HCT outcomes by integrating individual patient and treatment data with machine learning models. It provides a granular analysis of post-transplant survival and potential complications.

The tool's multiobjective analysis feature allows clinicians to assess various treatment paths and their respective trade-offs. By leveraging a multiobjective approach, easy filtering and navigation through various patient options is possible. This feature assists healthcare professionals in making decisions that not only address the clinical aspects but also consider the patient's risks and preferences. Moreover, the implementation of a Self-Organizing Map (SOM) analysis helps clinicians navigate patient options by clustering outcomes, revealing patterns that inform personalized treatment strategies and improve clinical decision-making.

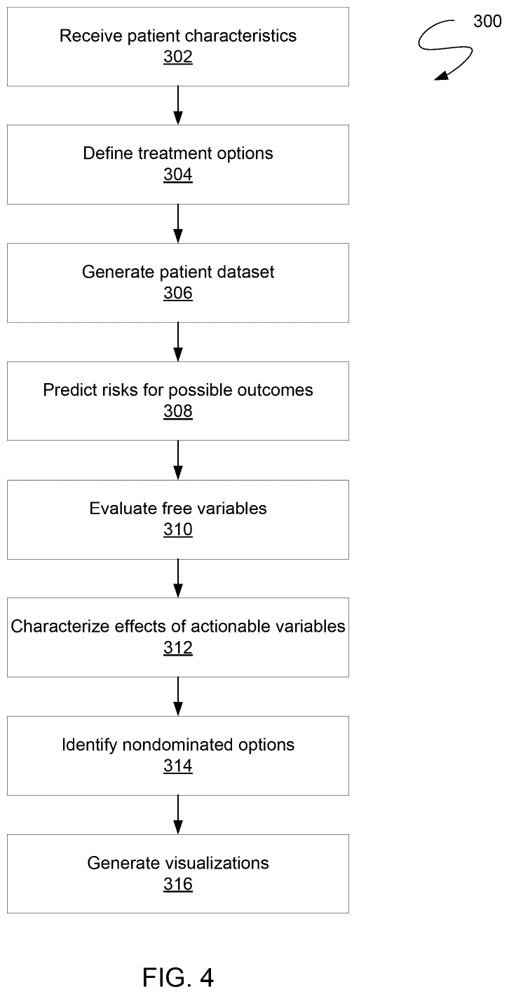

Referring now to FIG. 4, a flowchart of an example method 300 for performing the trade-off analysis is shown. Method 300 may be carried out by trade-off analyzer 214 of HSCT decision support system 200 of FIG. 3A.

At 302, a set of patient characteristics is received. Patient characteristics may be predefined and received locally or from a remote communication. Patient characteristics may be for a single patient, or several patients may be analyzed as a group. The set of patient characteristics may include selections of variables to be explored as independent variables and variables to be kept fixed.

At 304, a set of treatment options may be defined. Trade-off analysis involves enumerating and evaluating the possible treatment options for a given patient with known characteristics. Treatment options may be limited based on patient characteristics or available resources. Treatment options may be enumerated by a physician or another user, or may be determined by the system based on the set of patient characteristics and other data available, e.g., characteristics of available donors.

At 306, a patient dataset is generated. Enumeration of options produces a dataset including the patient characteristics and setting the variables so that some variables are fixed and other, e.g., the free variables, are seen in all possible combinations. While some variables, particularly recipient-specific variables, may be determined based on the patient dataset alone, enumeration of treatment options may introduce additional free or fixed variables to be received or determined by the system prior to analysis.

At 308, the patient dataset is analyzed with a set of outcome models, such as outcome models 204-212 of FIG. 3A. Separate models or sets of models may be provided for each outcome. The patient dataset is evaluated for a probability of the particular outcome which may be output as a risk score associated with that outcome. For example, for a time-to-event outcome of interest, an amount of months lost up to predetermined time is predicted.

At 310, the free variables of the patient dataset are evaluated based on the predicted outcomes from step 308. Step 310 may be performed using a multi-objective optimization framework and may be executed by a trade-off analyzer, such as trade-off analyzer 214 of FIG. 3A. Evaluation of free variables may be based on the predicted outcomes, and may output results indicating relative importance or impact of a given variable on the predicted outcome.

In embodiments, free variables may be evaluated using variable importance scores. Variable importance scores may be used to quantify how much individual features contribute, across a dataset, to predictions and may show the importance of predictor variables for each outcome analyzed. For example, the relative importance scores and a rank ordering of predictor variables may be different for different outcomes or may reveal overlap among one or more key predictor variables between different outcomes. Variable importance scores can reveal key factors in a patient's predicted outcome and whether, for example, this variable is actionable or fixed. Physicians may adjust their strategy based on their ability to make positive, or avoid negative, impacts in a patient's predicted outcomes.

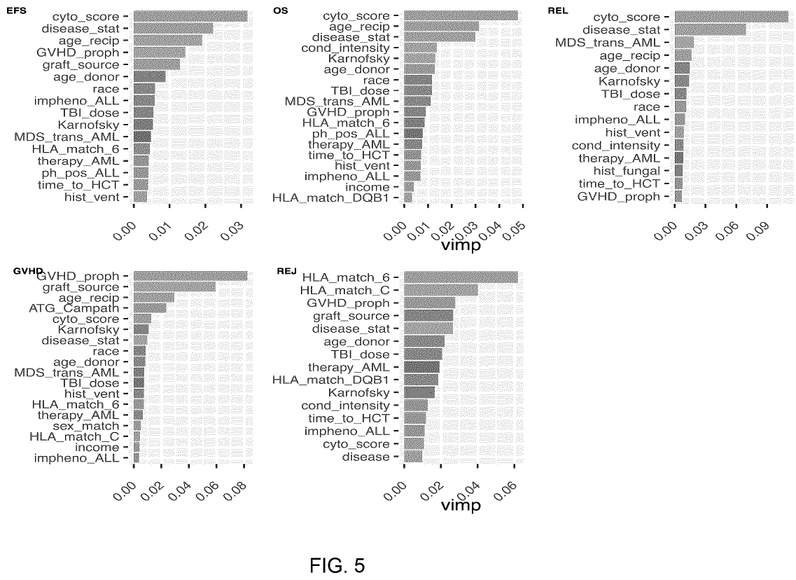

FIG. 5 presents an example of a visualization 400 of variable importance scores and illustrates how they can aide a clinician in evaluating outcomes. The example of FIG. 5 uses scores obtained from using RSF models. The importance of top predictor variables for each outcome analyzed are shown ranked from top to bottom. FIG. 5 illustrates that the ordering of the relative importance scores for predictor variables are different for different outcomes, although there is some overlap among the top variables. The lengths of the bars measure the importance of the variables for the calculation of risk scores. For instance, where GVHD prophylaxis is the most important predictor for GVHD, this variable does not appear to be important for relapse. Similarly, relative importance scores for the HLA matching scores seem are deemed the most relevant to rejection, but not to relapse.

As the HSCT and cellular therapy decision support system can present both a patient's mostly likely outcomes and the most influential variables in those outcome to a physician, the physician can better determine a course of action to adjust a patient's outcome in a desired direction. Variable importance scores may also be applicable in variable selection.

Free variables may be evaluated using a dependence analysis to quantify effects associated with one or more predictor variables. In examples, dependence estimates may be obtained using outcome models (such as outcome models 202-214) to predict outcomes for a dataset and then applying analysis of variance or Tukey's honest significance test (HSD) to analyze multiple comparisons of the predicted outcomes. The analysis of variance may include terms for controlling possible confounding factors, such as a patient's age or Karnovsky score.

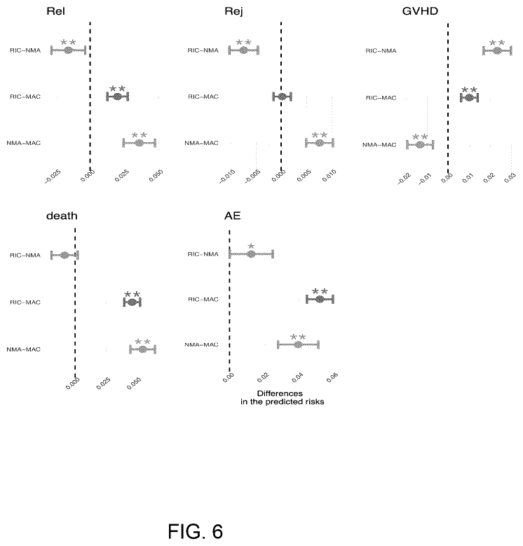

FIG. 6 presents an example of a visualization 500 of dependence analysis for the conditioning intensity variable on transplant outcomes. The example of FIG. 6 presents a comparison of the predicted risks for the different conditioning intensities. Displayed is the differences between the mean scores for the categories of the conditioning variable for different outcomes, the corresponding Tukey HSD based confidence intervals, and significance indicators. If a difference is negative, this means that the risk for the subtracted condition is higher than the risk associated with the condition that it is subtracted from. If a difference is positive, this means that the risk for the subtracted condition is lower than the risk associated with the condition that it is subtracted from. If a confidence interval does not include zero, this means that the difference is statistically significant. The outcomes are rejection, relapse, GVHD, death, and adverse outcome (AE).

In this example, for each outcome lower risks scores are preferable. The stars above the bars indicate the significance of the differences (*p<0.05, **p<0.01, ***p<0.001). In the example of FIG. 6, Myeloablative conditioning (MAC) is shown to be the best option for the risks involved with rejection, relapse, and OS, but for GVHD and EFS, patients that have non myeloablative conditioning (NMA) are at the lowest risk.

In examples, free variables may be evaluated using exponentiated coefficient estimates and related confidence intervals and p-values for a selected subset of predictor variables for each of the outcomes analyzed. Exponentiated coefficients can be interpreted as the multiplicative effect of the predictor on the hazard rate of an individual for one unit change in the predictor variable. If the hazard ratio for a predictor is close to 1 then that predictor does not affect survival. If the hazard ratio is less than 1, then the predictor is protective, and if the hazard ratio is greater than 1, then the predictor is associated with increased risk.

Referring back to FIG. 4, at 312, effects of actionable variables are characterized. At 314, nondominated options are identified from among the enumerated treatment options. Together, steps 312 and 314 evaluate impacts associated with adjustments to actionable variables and what combination of adjustments yield desired results. Trade-off analysis studies the trade-offs related to the HSCTs for a patient over varying values of a set of actionable variables. When the effects of the actional variables are characterized, at 312, different values of one or more actionable variables are adjusted across a range, such as through a Pareto optimization process. Following this optimization, one or more nondominated options may be identified, at 314.

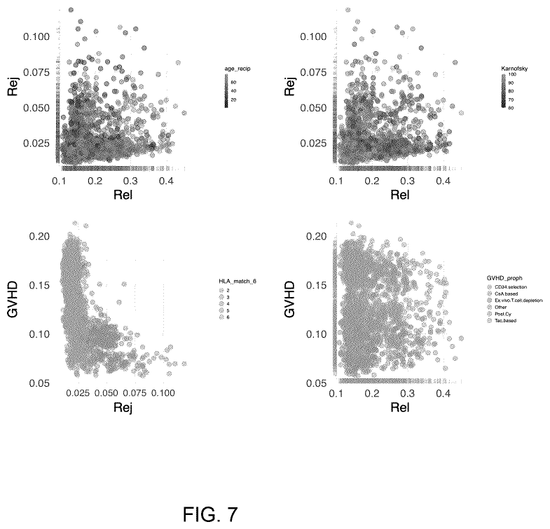

For example, FIG. 7 presents a set of example visualizations including plots 600a-d illustrating the effects of several predictor variables on the HSCT, two outcomes at a time. This can be useful for studying the simultaneous effect of variables on different outcomes in the trade-off analysis.

In the example of FIG. 7, an expected number of months lost due to different causes as associated with several predictor variables for a population of HSCTs is shown. Such plots 600a-d may be useful to visualize the simultaneous effect of these variables on different outcomes. The plots 600a-d in this example indicate that patient age has a negative impact on both rejection and relapse, with a greater effect on relapse. Higher Karnovsky scores are associated with lower risks of rejection and relapse. Type of GVHD treatment affects GVHD risks, but not relapse risk; cyclophosphamide-based treatments are more effective than tacrolimus-based treatments. HLA matching at 6 loci affects predicted rejection risks, but not GVHD risk.

Trade-off analysis and related plots can be used to examine how the predictors affect the outcomes, and what changes to actionable predictors can yield desired outcomes. More importantly, the plots 600a-d and other visualizations can be used to identify a set of non-dominated treatment options for a given patient. Steps 312 and 314 of FIG. 4 may involve generation of summary plots, figures, or statistics, such as colored 2 or 3-dimensional scatter plots, SOM plots, cluster analysis, plots of predictions of survival functions and competing risks functions, estimates of median survival time, etc. Steps 312 and 314 may be performed in parallel or in sequence. Steps 312 and 314 may be performed iteratively, for different combinations of actionable variables and outcomes.

Continuing discussion of FIG. 4, at 316, one or more of the plots, figures, or statistics generated by steps 312 and 314 may be displayed as a visualization to a clinician, such as a physician or other user, to aid in decision-making. In embodiments, the visualization may be responsive to manipulations by the user. For example, the user may have the to adjust some or all of the free variables to observe the effects of these variables on the predicted outcomes.

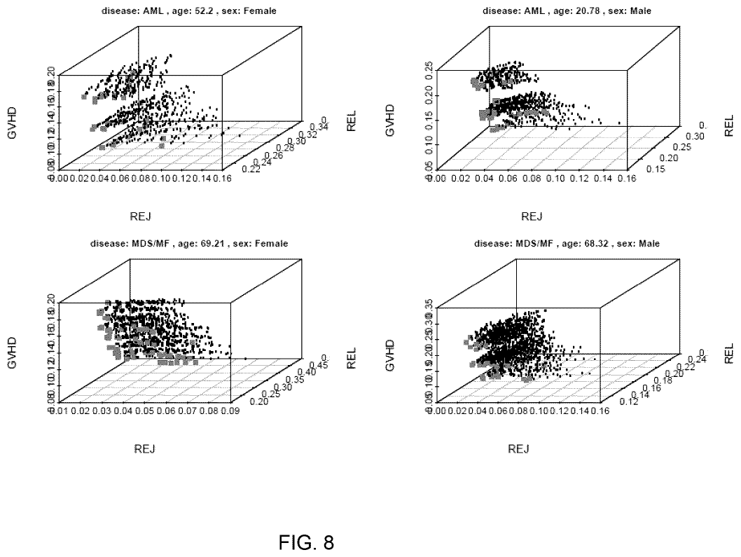

FIG. 8 presents a plurality of charts, illustrating examples of trade-off plots 700a-d for 4 patients. The example of FIG. 8 uses trade-off plots generated by changing the values of the covariates of the variables conditioning intensity, graft source, GVHD prophylaxis, TBI does, donor relatedness, donor age, and a set of HLA matching variables. Other variable values were fixed, and the risk score related to each of the five HCT outcomes was predicted using outcome models, such as outcome models 204-212 of FIG. 3A.

In embodiments, a physician or other user may set database criteria for risk assessment or trade-off analysis. In the example of FIG. 8, all categorical variables and the numerical variables values were restricted to the observed values in the part of the data which involve patients with the same disease as the current patient and also patients within 5 years of the age of the current patient. Donor age was set to 25%, 50% and 75% percentiles of the restricted donor age values. This data-based method is an example method to eliminate unfeasible or impractical treatment options to an extent. While putting the trade-off analysis into practice, the proposed treatment combinations may also be evaluated for feasibility and/or practicality. For example, availability of donors with a particular HLA match given a patient and a donor registry can be used to restrict donor related characteristics such as donor age and HLA match in the trade-off analysis to characteristics of available donors.

The blue points in FIG. 8 are showing the non-dominated options for the competing risk outcomes of rejection, relapse, and GVHD. The red point is the de facto for the patients. The black points are the dominated options.

In the example shown, the Pareto surface represented by the blue dots characterizes the trade-offs among the different risks for each of the patients. Each patient has a slightly different profile for risks. The plots 700a-d show that, in most cases under these example considerations, the de facto conditions were dominated, e.g., there were options that were predicted to improve the patient's outcome with respect to at least one of the risks without increasing the risks for the other outcomes. Unless the risks associated with all the outcomes are well-correlated, a multitude of non-dominated options may appear instead of a single one.

The final decision on the treatment will involve many factors such as the patient's preferences, the physician's expertise, the availability of resources, and the feasibility of the treatment options. Trade-off analysis provides a tool to aide in making the final decision by providing the physician with visualizations of likely outcomes to different treatment options.

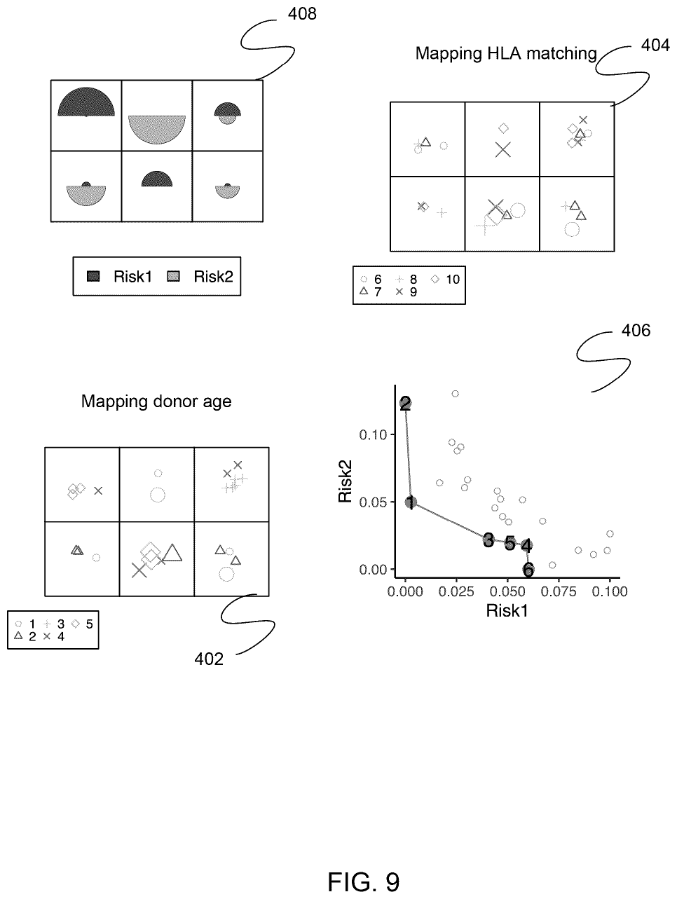

Referring now to FIG. 9, another example output of a trade-off analysis is shown. In this example, a set of free variables is defined including donor age 802 (values 1,2,3,4,5 corresponding to 5 ordinal categories of patients' ages from youngest to oldest) and HLA matching score 804 (6,7,8,9,10 out of 10 HLA matching scores). All possible combinations of these two factors yields 25 possible treatment options. These values and a set of patient characteristics are used to predict risk scores for two outcomes for all 25 possible treatment options.

The scatter plot 806 of the estimated risks for the two different outcomes and the Pareto analysis of these options indicate that only 6 out of these 25 options are non-dominated (the red points on the scatter plot) and all the other treatment options are dominated by these 6, meaning that improvement over the dominated options is available by selecting one of the 6 non-dominated options.

SOM analysis 808 of these 25 treatment options on the “value plot” provides a representation of the trade-off between the two risks. Comparing the “value plot” to the corresponding plots of HLA matching score 804 and donor age 802 provides a visualization of how these risks change based on different treatment options. Larger points on the mapping plots of donor age 802 and HLA matching score 804 are the non-dominated treatment options.

Each point in each of the four SOM plots is an option for the patient. Each square in the values plot 408 and the mapping plots 802 and 804 represent a cluster of the choices. The clusters are formed such that the values within a cluster is homogeneous as compared to the values across different squares. Nearby squared in the value plot are similar to each other in terms of the values.

Together the plots of FIG. 9 reveal the trade-off between the HSCT risks in this example is accompanied by the choices between the ‘young donor-low match’ versus ‘older donor-high match’ treatments.

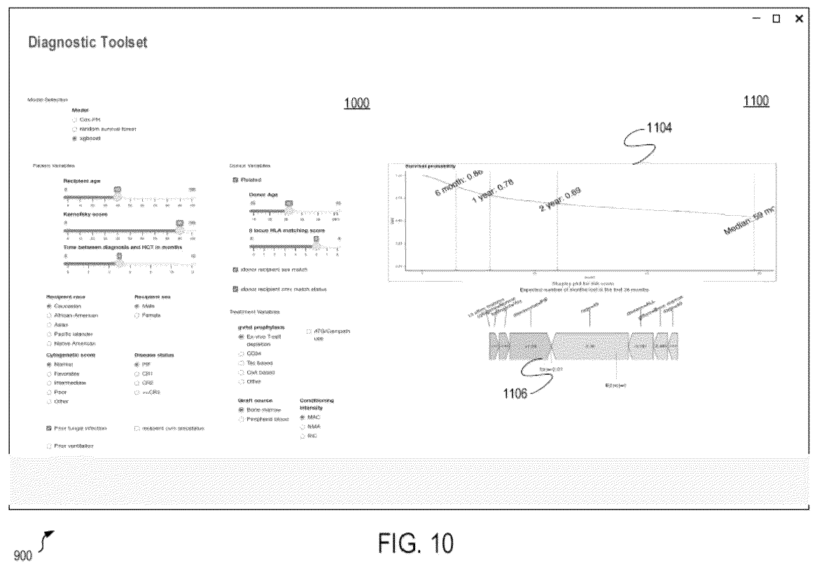

Referring now to FIG. 10, an example user interface 900 for the HSCT decision assistance tool is shown. The user interface 900 generally includes a toggle board 1000 as well as a modeling visualization 1100. Generally speaking, the user interface 900 may provide a display of one or more outputs of the HSCT decision assistance tool as described in greater detail above. User interface 900 may be static or interactive, and may be responsive to any known input feature such as a mouse, a touch screen, a keyboard, voice commands, etc.

In the example shown, the toggle board 1000 provides a display of one or more variables evaluated by the HSCT decision assistance tool. Toggle board 1000 may display some or all variables used in risk assessment and trade-off analysis, including patient variables, donor variables, and treatment variables. The toggle board 1000 may also allow a user to select a particular model for use in predictive analysis.

In embodiments, toggle board 1000 may display only actionable variables. Actionable variables may be associated with interactive features on toggle board 1000. For example, variables associated with a range, such as donor age, may be associated with a slider bar. The slider can be adjusted within the range, allowing a physician to review changes to the patient's outcomes in response to changing variables.

The modeling visualization 1100 can include, in the embodiment shown, a survival plot 1104, and a Shapely plot 1106.

Survival plot 1104 provides a visualization of a time to an event. Example survival plot 1104 depicts an OS plot, but time to event plots of other outcomes are contemplated and, in embodiments, a physician or other use may view one or more survival plots at a time, or select the outcome or event plotted. In embodiments, survival plot 1104 may represent a viewing space in the user interface and the user may select the visualization to be displayed. For example, any of the plots of FIGS. 5-9 may be displayed.

Shapely plot 1106 represents a visualization of explainable AI features of the HSCT decision assistance tool. The baseline for Shapley values is the average of all predictions. In the plot, each Shapley value is an arrow that pushes to increase (positive value) or decrease (negative value) the prediction. These forces balance each other out at the actual prediction of the data instance. Positive SHAP value means positive impact on prediction, leading the model to predict 1. Negative SHAP value means negative impact, leading the model to predict 0.

Shapely plot 1106 provides a physician or other user with some insight into the basis for the predictions output by the outcome models and the trade-off analyzer, and thus provides the user with data by which to gauge their confidence in and reliance on the predictions provided. Further, Shapely plot 1106 can be informative of the relative weight and impact of variables.

In alternative examples of the user interface 900, other types of graphical features may be presented. For example, in some instances, more or fewer types of models may be presented for selection; in some instances, the model used for analysis may be automatically selected based on organizational preference, historical accuracy, or other factors. Additionally, more or fewer variables may be presented for selection. Additionally, regarding the modeling visualization 1100, in some instances a recommended treatment is presented based on Pareto optimization, alongside one or more factors that are most indicative of selection of the optimal or recommended treatment. Such a recommended treatment may be presented either in conjunction with graphical analysis including the survival plot 1104 and Shapely plot 1106, or as an alternative thereto. Still further, other types of graphical representations may be provided as well (e.g., a plot of a Self-Organizing Map, or SOM, or the like).

In addition to the toggle board 1000 and modeling visualization 1100, it is noted that a user interface 900 presented to a clinician (e.g., clinicians 16 as described above) may present a variety of other diagnostic toolsets. For example, in some instances, the HSCT decision assistance tool may be presented within a set of tools that can also provide comparative patient outcome records for review and display, outcome prediction for HLA matching, including an aggregate HLA matching tool, and the like. Such tools are intended as exemplary, and not limiting.

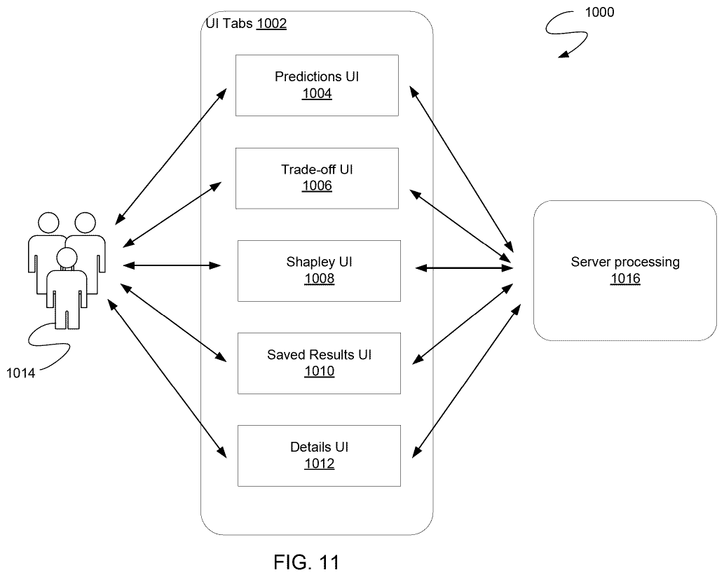

Referring now to FIG. 11, a server application flowchart depicts an example operational core 1000 driving the user experience and backend functionality. The design of the server application ensures that user interactions are not only intuitive but also effective in translating complex data into meaningful clinical outcomes. The flowchart is an example blueprint that illustrates the integration of user inputs with robust data analytics, culminating in a highly responsive and insightful tool for clinicians.

At the core of the user experience are a set of user interface (UI) tabs 1002. In this example, UI tabs 1002 which serve as the primary touchpoints for users 1014, enabling them to interact with the application's features and functionalities. The example of FIG. 11 highlights the example UI tabs—Predictions UI 1004, Trade-off UI 1006, Shapley Analysis UI 1008, Saved Results UI 1010, and Details UI 1012—which are the main interface elements guiding users through data input, analysis, and result interpretation. This flowchart serves as a blueprint showcasing how user inputs drive the backend processes, ensuring a responsive and insightful analytical experience. By bridging user interaction with complex data processing, it underscores the application's role in facilitating accessible, informed clinical decision-making in HSCT.

Predictions UI 1004 allows users to input data and receive predictive analyses. This interface translates complex variables into tangible predictions that a user 1014 may apply to guide therapeutic choices.

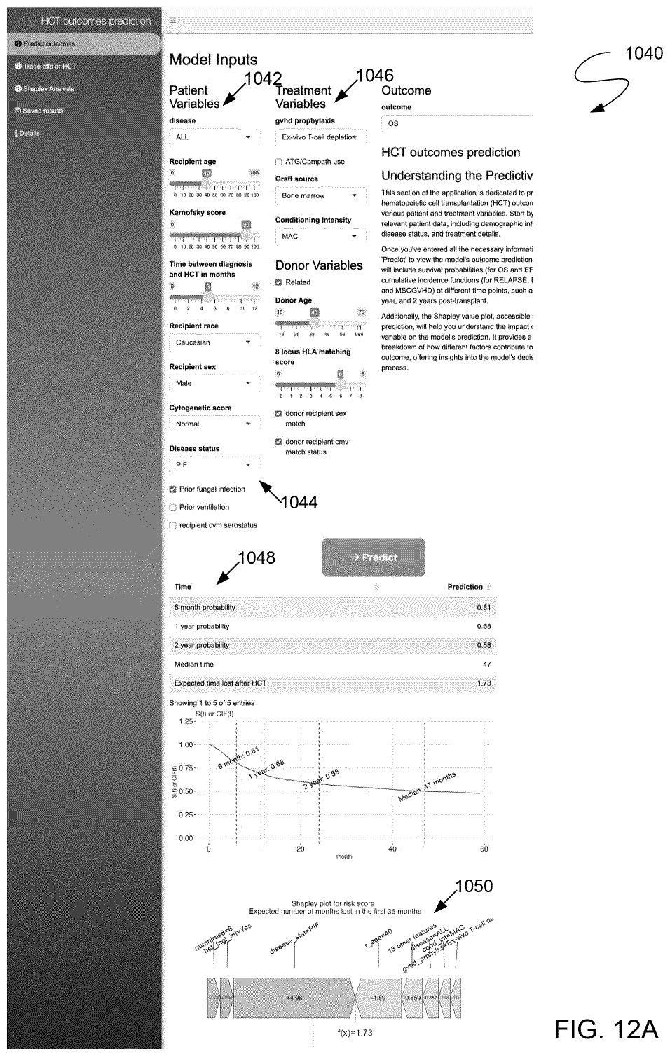

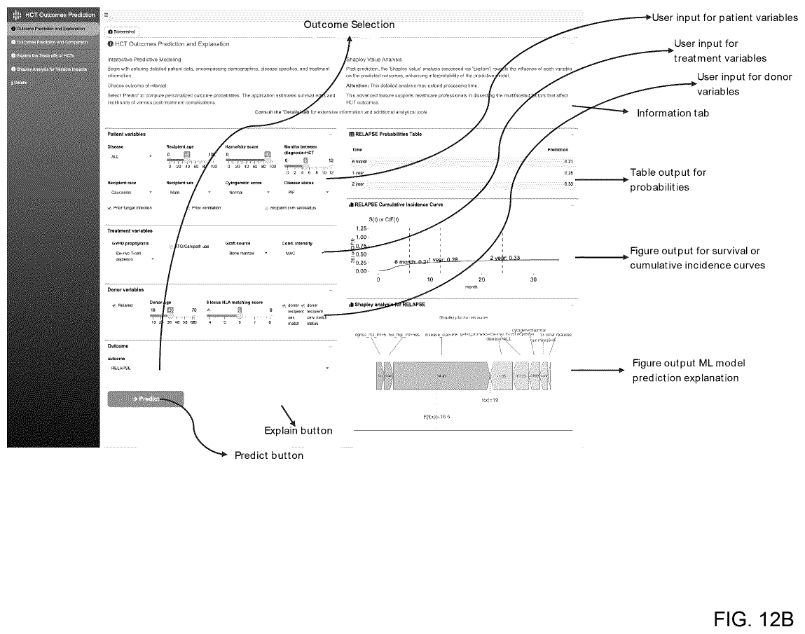

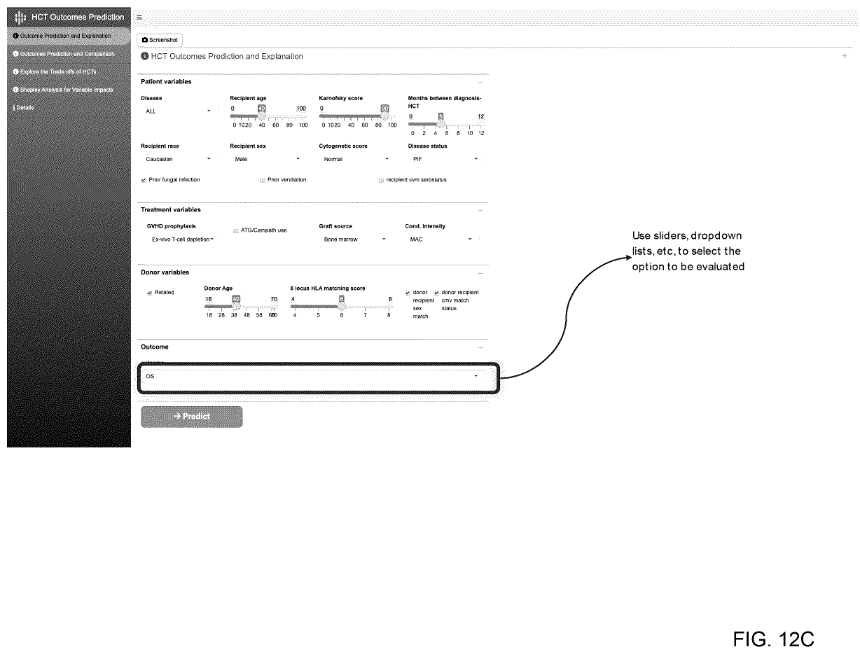

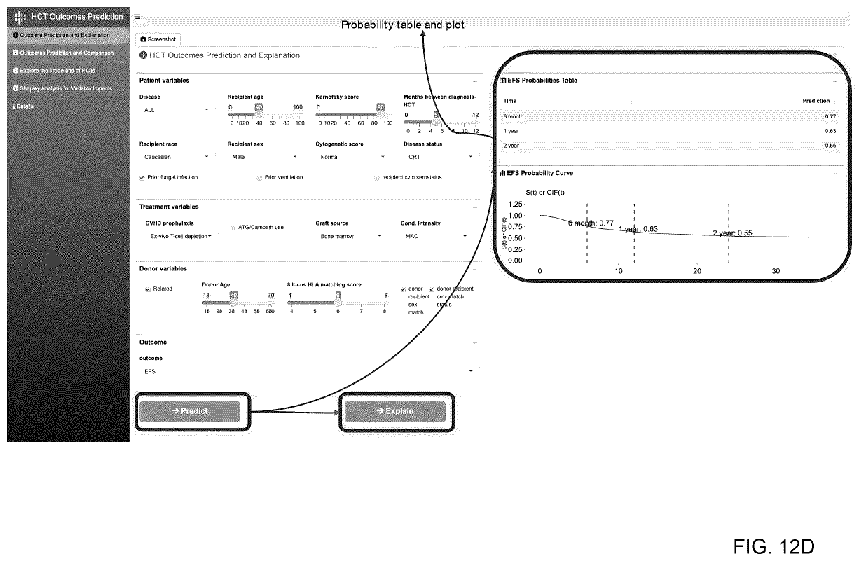

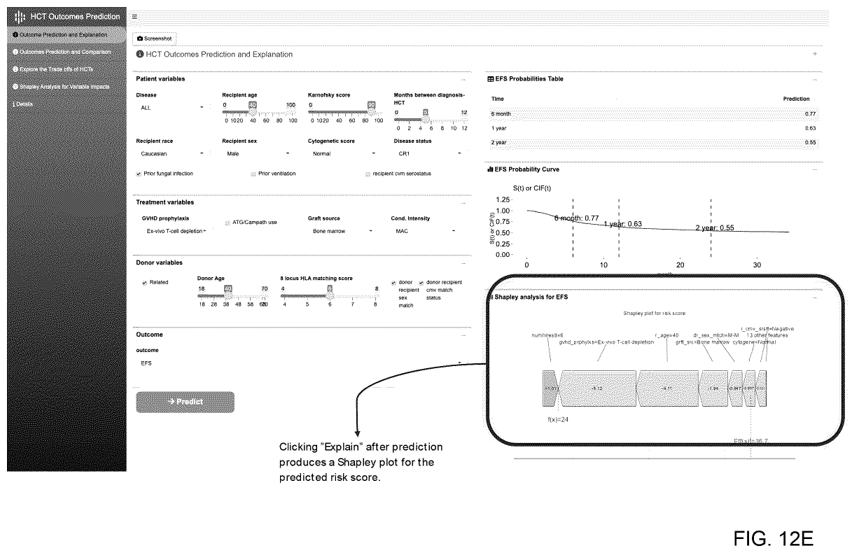

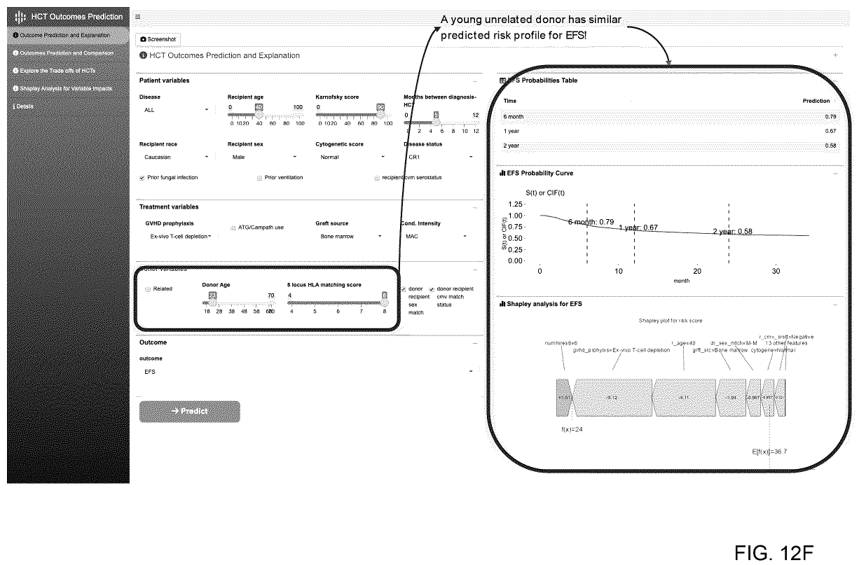

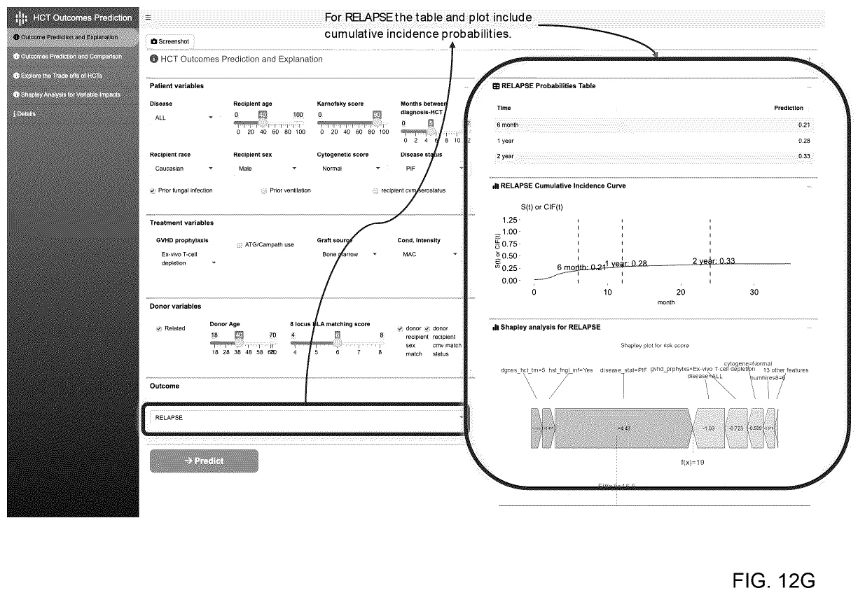

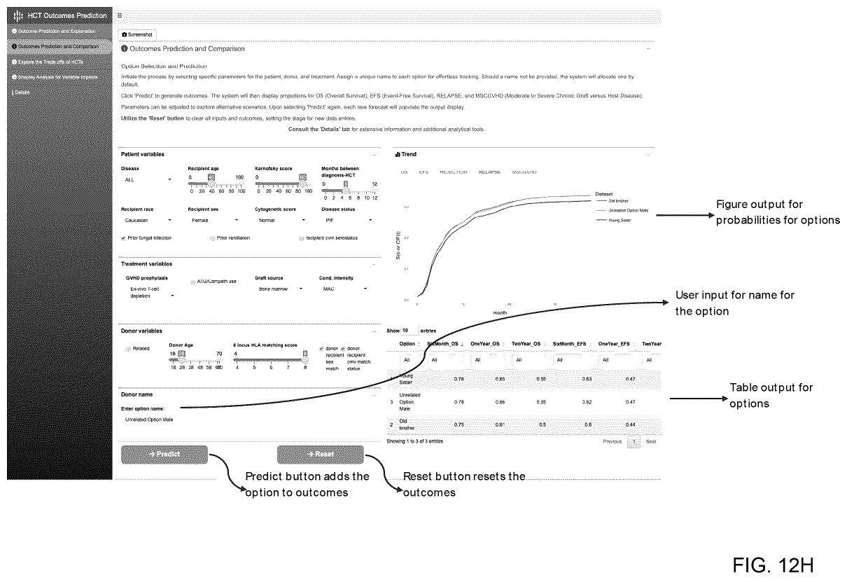

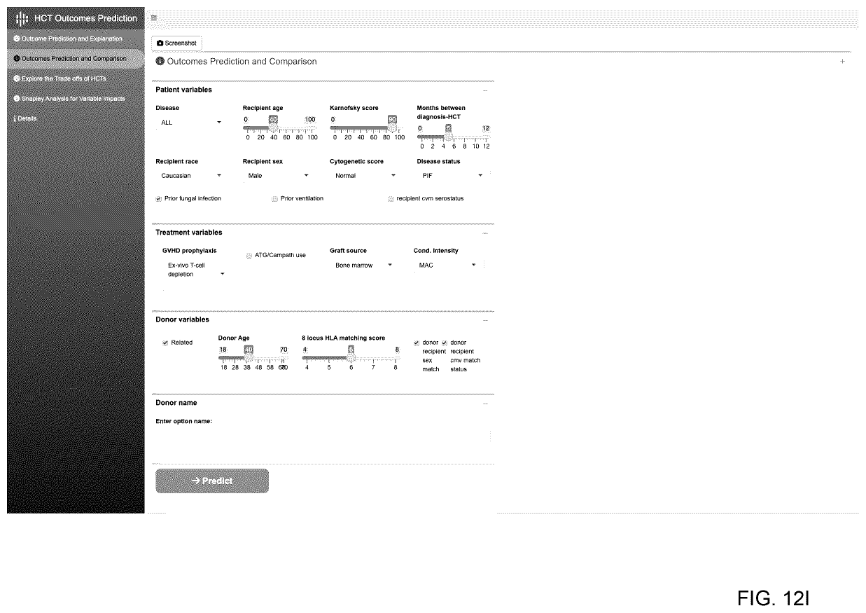

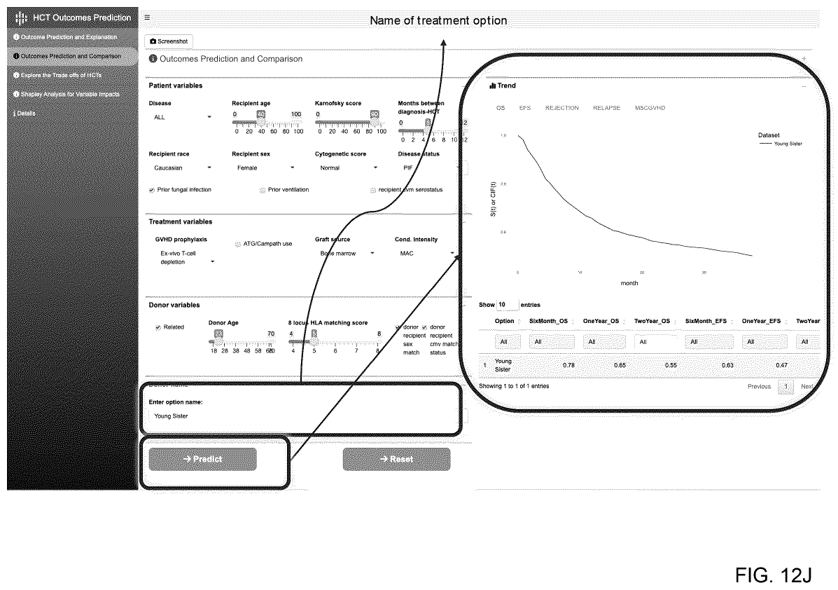

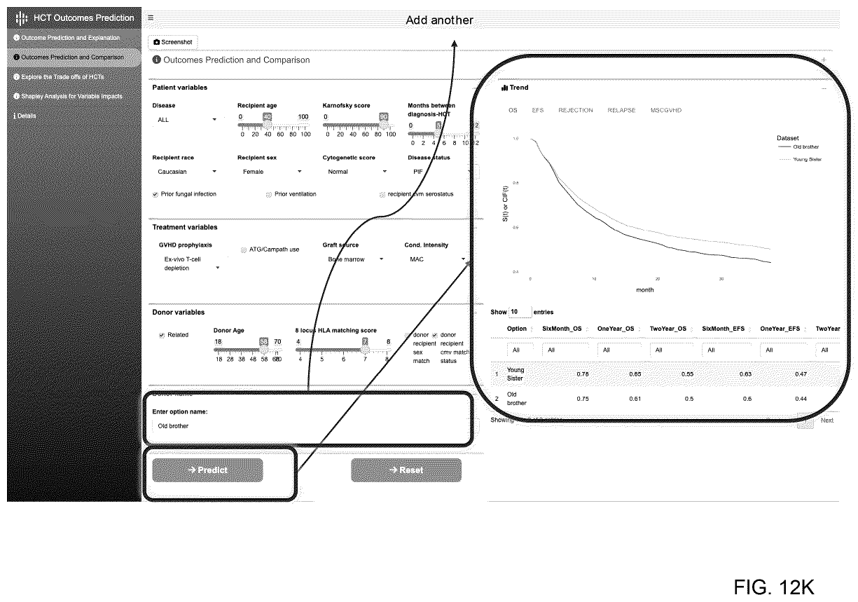

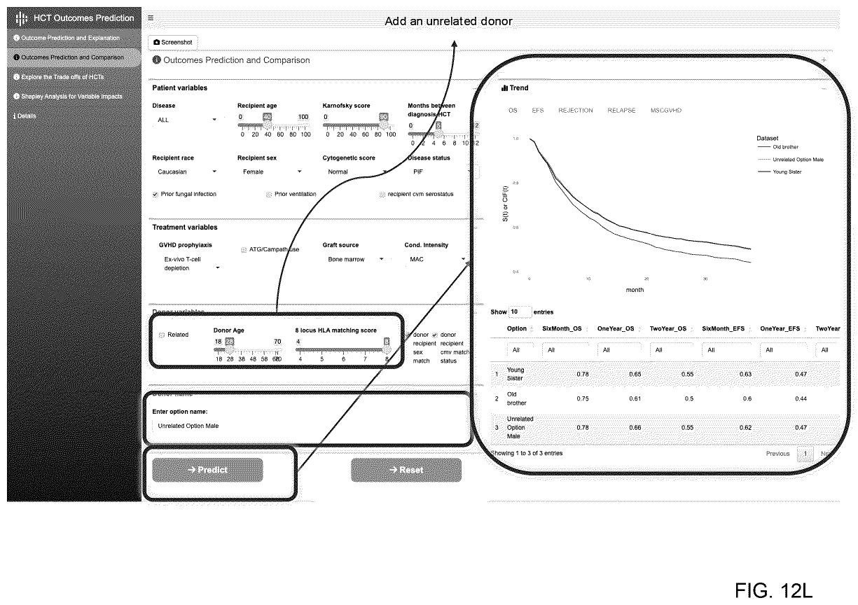

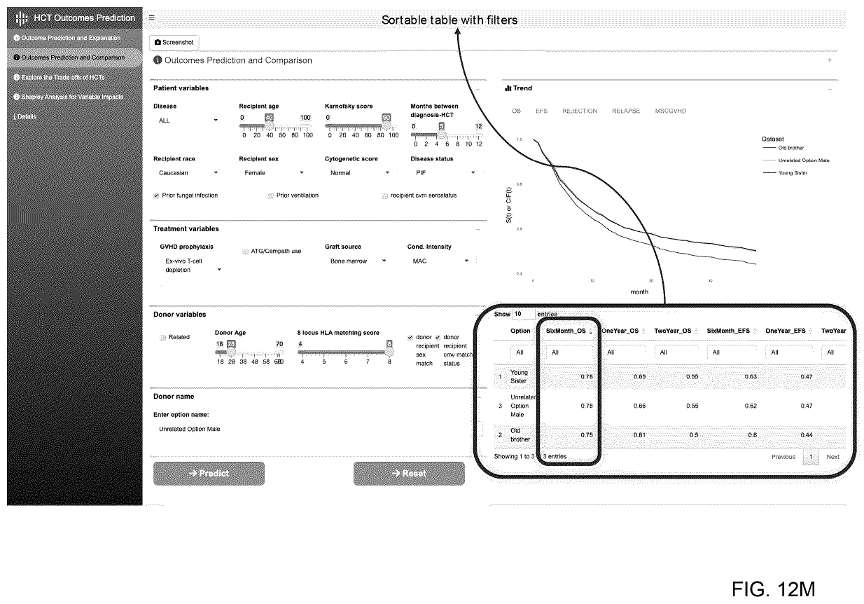

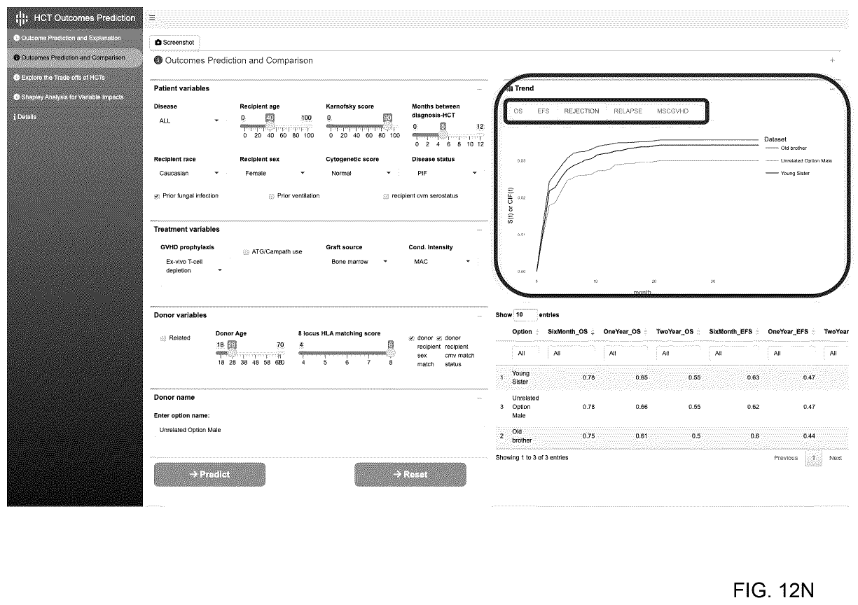

FIG. 12A is an example graphical user interface (GUI) 1040 depicting a predictions UI 1004 of FIG. 11. This interactive module is designed to forecast HSCT outcomes by integrating a range of patient-specific data and treatment variables. The user-friendly interface allows for the seamless entry of, for example, demographic information 1042, disease status 1044, and treatment details 1046 (for additional detail, see FIGS. 12B, 12C, 12F-12I and their associated description below). Upon submission, the system employs advanced machine learning algorithms (e.g., models 202-212 of FIG. 3Aa) to generate outcome probabilities 1048 and cumulative incidence functions for critical post-transplant metrics(for additional detail, see FIGS. 12B, 12D, 12H, 12J-12N and their associated description below). The integration of a Shapley value plot 1050 demystifies the model's rationale by quantifying the influence of each variable on the predicted outcomes, thereby augmenting the clinician's interpretative capabilities and reinforcing the decision-making process (for additional detail, see FIGS. 12E-12G and their associated description below).

FIGS. 12B-12G are further examples of GUI 1040 of FIG. 12A, depicting various example explanation operations of predictions UI 1004. FIG. 12B is another example of GUI 1040 of predictions UI 1004 identifying inputs and explanation output. Inputs include patient variables, treatment variables, donor variables, and selection of the outcome to be explored. Outputs include probabilities, survival or cumulative incidence curves, and an explanation for machine learning prediction (e.g., Shapley plot). Interaction points, such as prediction and explanation buttons, and information tab are also shown. FIG. 12C is another example of GUI 1040 of predictions UI 1004 identifying outcome selection options. In the example of FIG. 12C, an example of selecting an outcome type to be explored. In this example, a dropdown menu is used to select overall survival outcomes. FIG. 12D is another example of GUI 1040 of predictions UI 1004 demonstrating a probability table and plot. FIG. 12E is another example of GUI 1040 of predictions UI 1004 demonstrating a Shapley plot. FIG. 12F is another example of GUI 1040 of predictions UI 1004 demonstrating predictions responsive to changing inputs. FIG. 12G is another example of GUI 1040 of predictions UI 1004 demonstrating prediction outputs for a relapse outcome.

FIGS. 12H-12N are further examples of GUI 1040 of FIG. 12A, depicting various example comparison operations of predictions UI 1004. FIG. 12H is another example of GUI 1040 of predictions UI 1004 demonstrating inputs and predictive outputs for compare options. Additional inputs may include donor categories. Predictive outputs include comparative figures, such as graphs, and tables of the input options, each of which may be added by selecting a button (e.g., Predict button). FIG. 12I is another example of GUI 1040 of predictions UI 1004 demonstrating the GUI appearance prior to option selection. FIG. 12J is another example of GUI 1040 of predictions UI 1004 demonstrating the GUI appearance with one option selected. FIG. 12K is another example of GUI 1040 of predictions UI 1004 demonstrating the GUI appearance comparing two selected options. FIG. 12L is another example of GUI 1040 of predictions UI 1004 demonstrating the GUI appearance comparing three selected options. FIG. 12M is another example of GUI 1040 of predictions UI 1004 demonstrating operations associated with comparison table output. Example operations include sorting and/or filtering the table. FIG. 12N is another example of GUI 1040 of predictions UI 1004 demonstrating operations with comparison graph output. Example operations include selection of various outcomes by which to compare the selected options.

Trade-off UI 1006 facilitates the exploration of different treatment paths and their outcomes. Trade-off UI 1006 encapsulates the system's ability to navigate the complexities of HSCT outcomes, allowing clinicians to visualize and weigh the potential impact of varying treatment paths, enhancing patient outcomes through strategic analysis.

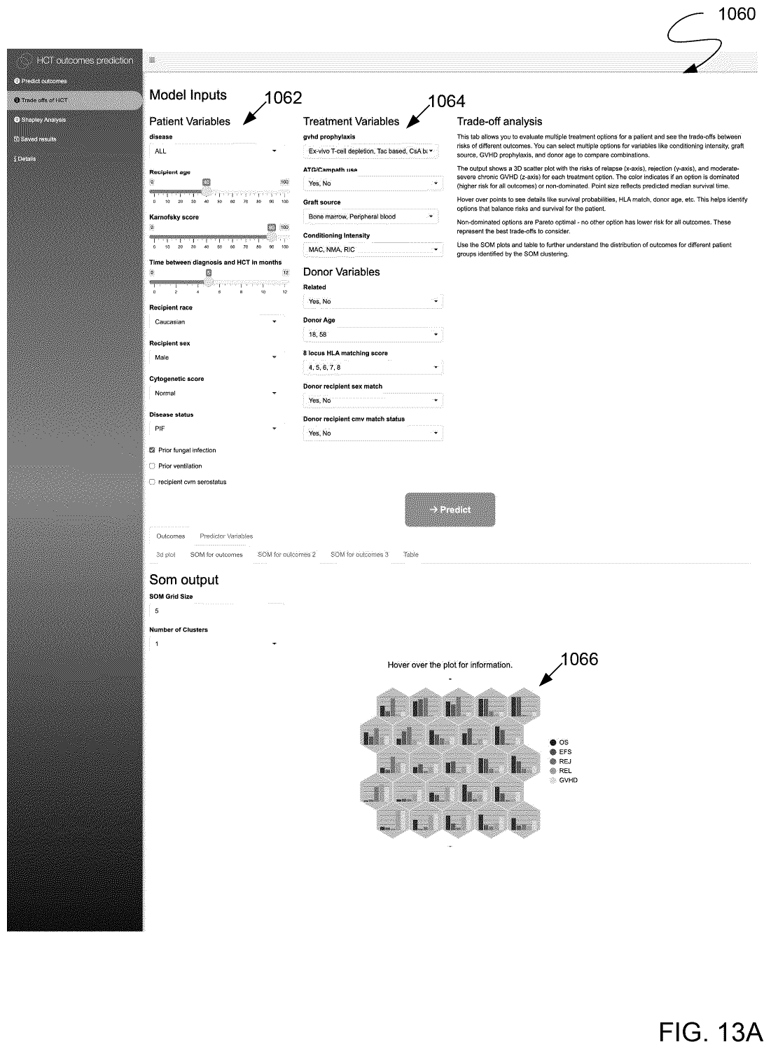

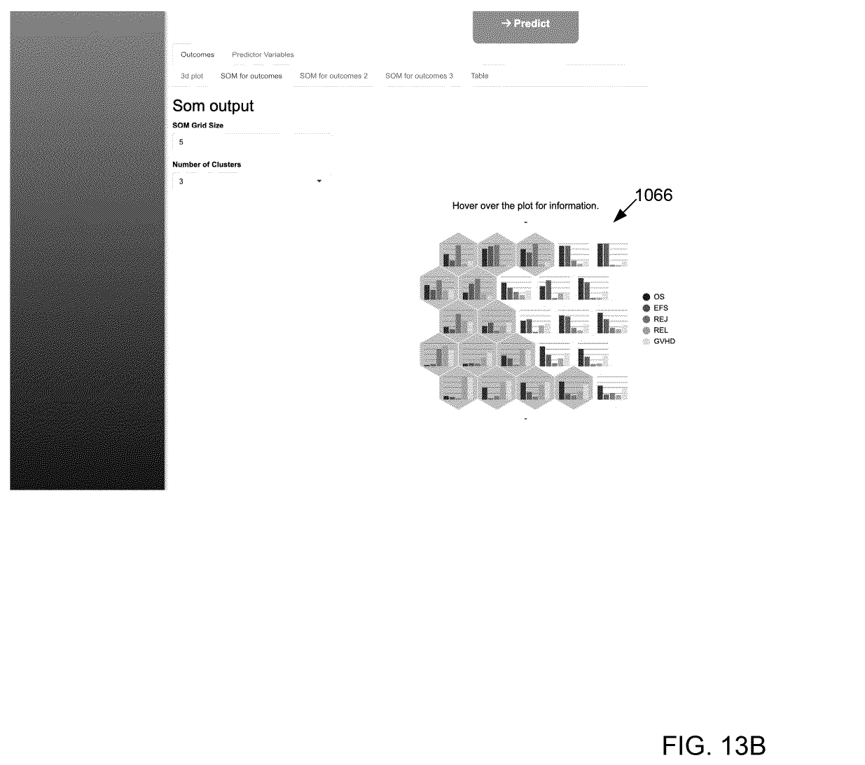

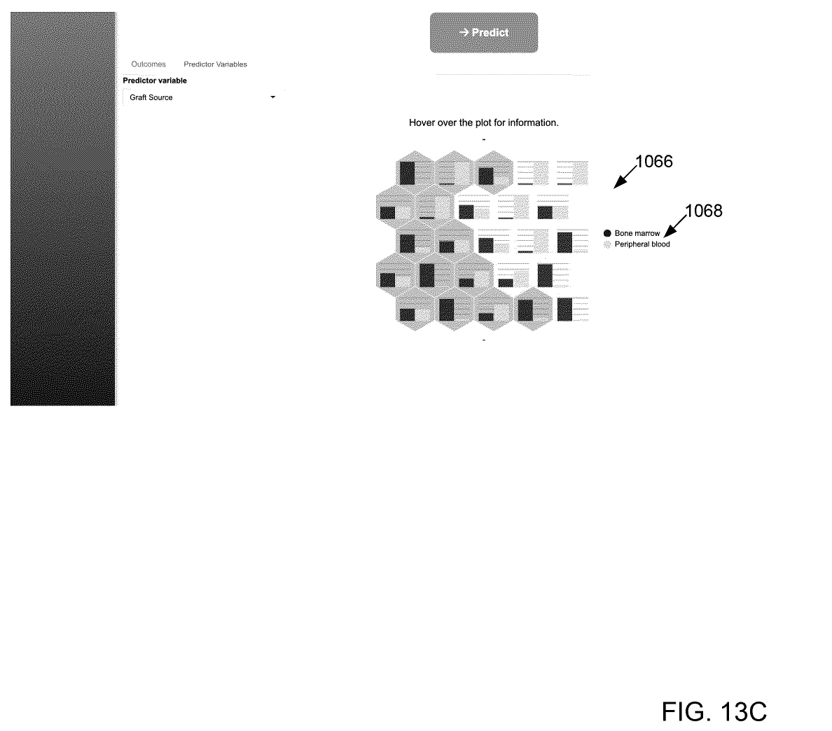

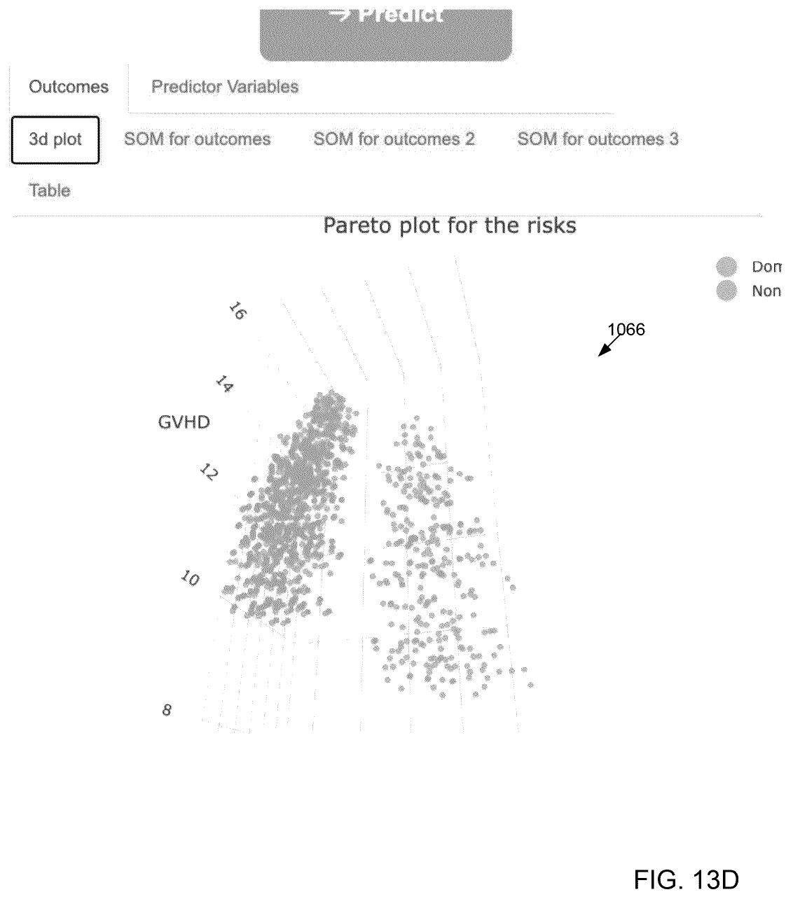

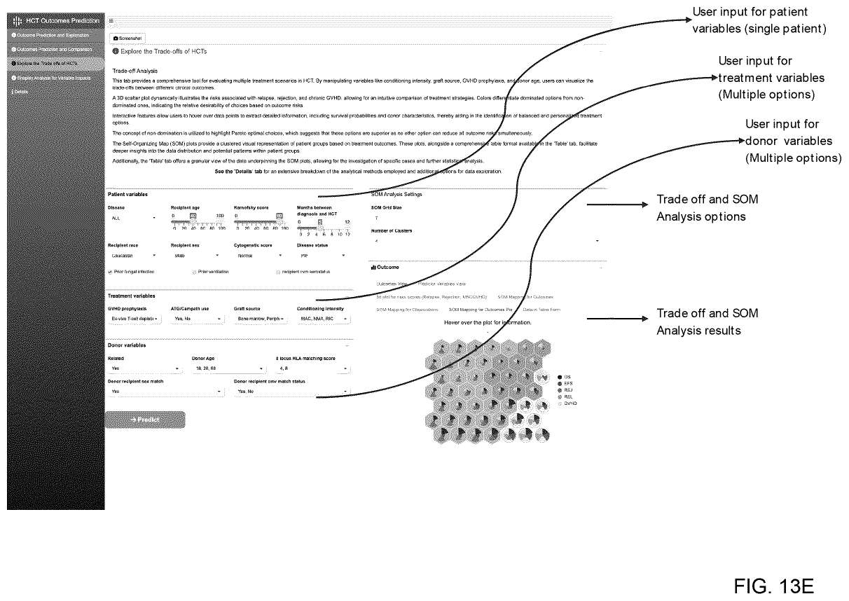

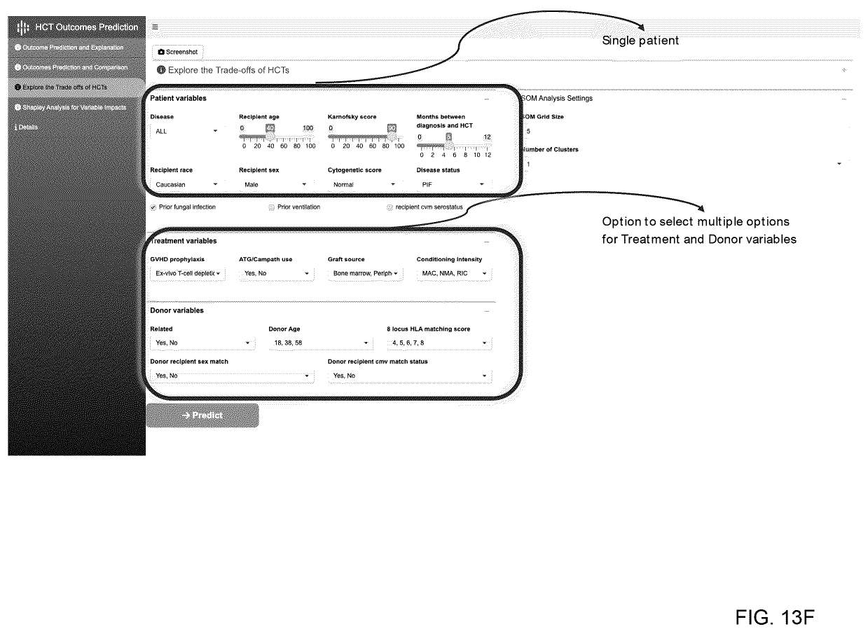

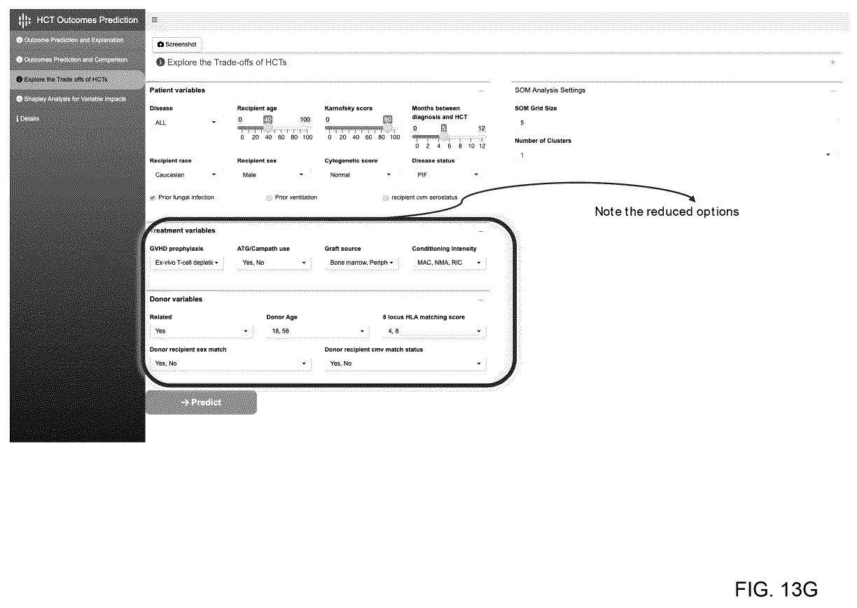

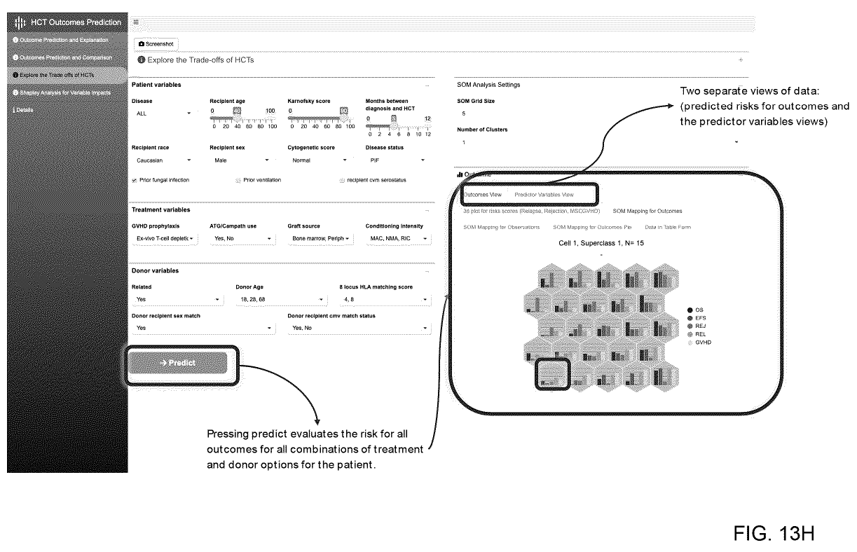

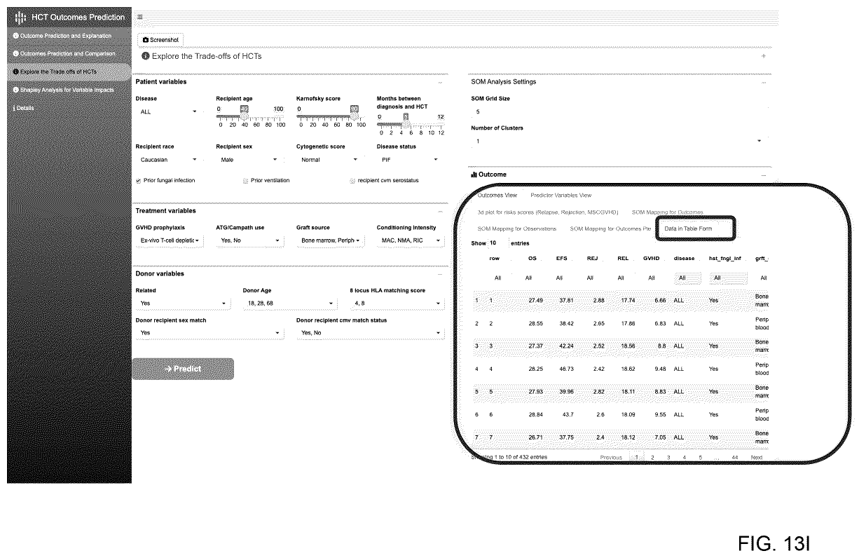

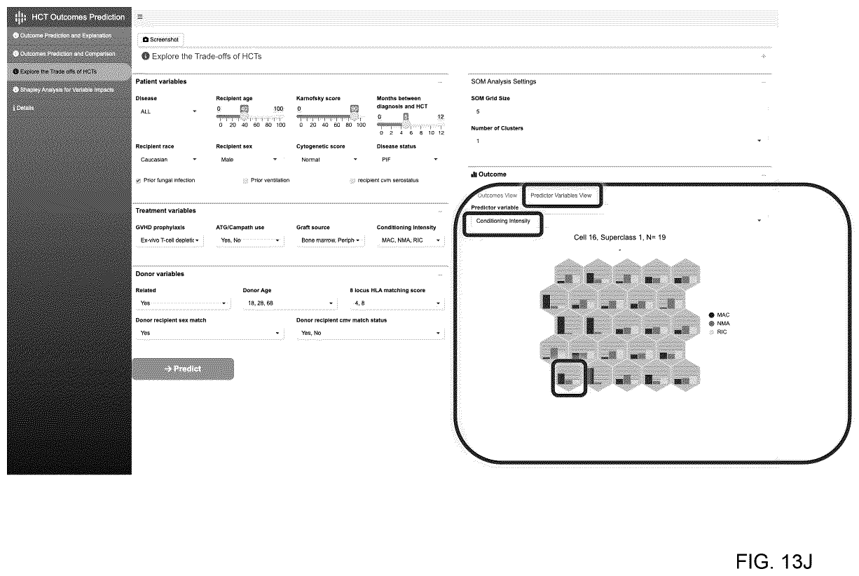

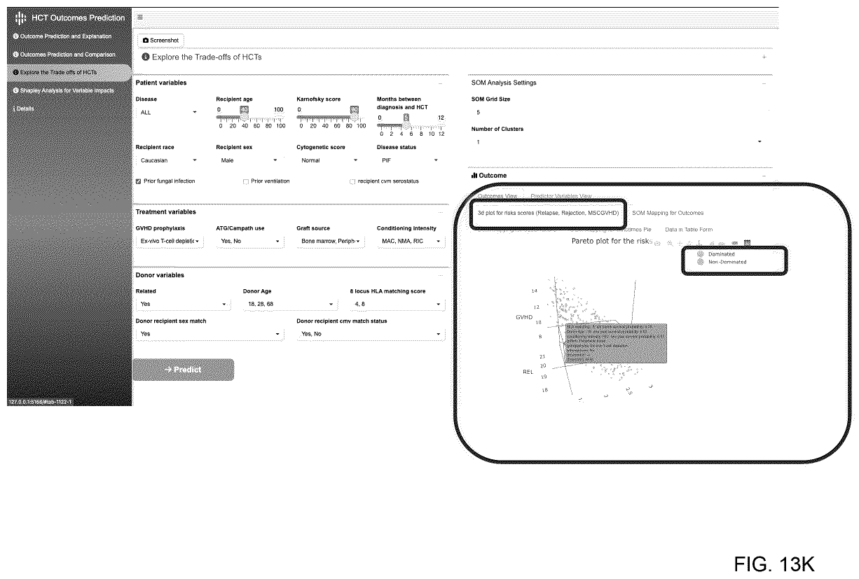

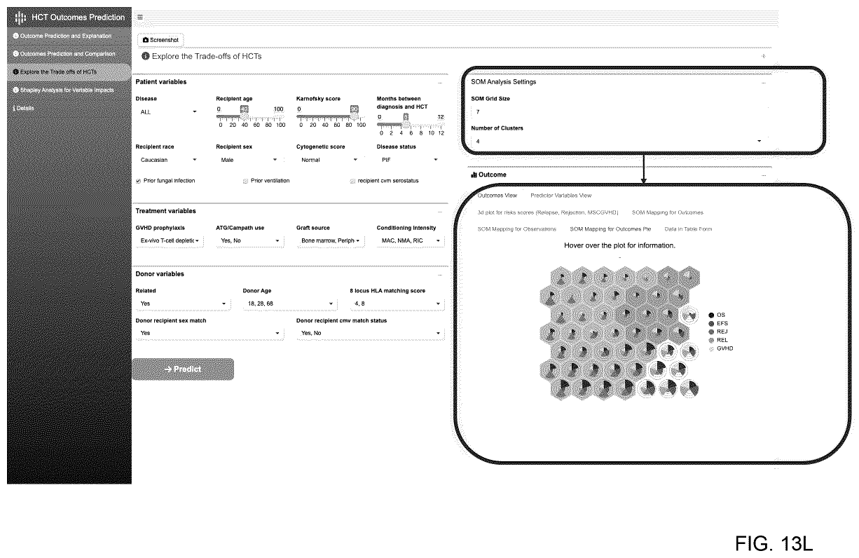

FIG. 13A is an example GUI 1060 depicting a trade-off UI 1006 of FIG. 11. This analytical interface enables comparison of multiple HSCT treatment strategies. Clinicians can assess the interplay of various factors based on both patient variables 1062 and treatment variables 1064, such as conditioning intensity and graft source, to discern the balance between differing outcome risks (for additional detail, see FIGS. 13E-13G and their associated description below). The visual output 1066, rendered as a 3D scatter plot or self-organizing map (SOM) (as shown in this example), delineates risk levels of relapse (REL), rejection (REJ), and chronic GVHD across treatment permutations(for additional detail, see FIGS. 13E and 13H-13L and their associated description below). By examining these multidimensional data points, clinicians can pinpoint Pareto optimal choices, those that strike the best balance without increasing other risks, and use this to tailor patient-specific treatment plans.

FIG. 13B illustrates additional details of the visual output 1066 of trade-off GUI 1060 of FIG. 13A. The SOM plot depicted by visual output 1066 in this example offers a multi-dimensional visualization of predicted HSCT outcomes, enabling the identification of patient treatment options with similar projected results (for additional detail, see FIGS. 13H, 13J, and 13L and their associated description below). This powerful analytical tool assists in discerning the optimal patient specific treatments by clustering outcomes, underscoring the system's capacity to synthesize complex data into actionable insights.

FIG. 13C illustrates other additional details of the visual output 1066 of trade-off GUI 1060 of FIG. 13A. In this example, the SOM analysis extends its utility by correlating the predicted HSCT outcomes with the actionable variables 1068 involved in treatment decisions (for additional detail, see FIG. 13J and the associated description below). Such correlative mappings are useful to clinicians for assessing the most effective interventions based on individual patient profiles.

FIG. 13D illustrates other additional details of the visual output 1066 of trade-off GUI 1060 of FIG. 13A. In this example, visual output 1066 presents a 3D scatter plot visualization which illustrates the complex interplay of predicted HSCT outcomes, for example with a color-coded scheme to differentiate between dominated and optimal treatment options (for additional detail, see FIG. 13K and the associated description below). Interactive elements provide additional patient-specific information, aiding clinicians in navigating the intricate landscape of HSCT treatment planning.

FIGS. 13E-13L are further examples of GUI 1060 of FIG. 13A, depicting various example operations of trade-off UI 1006. FIG. 13E is another example of GUI 1060 of trade-off UI 1006 demonstrating inputs and outputs of the UI. Example inputs include patient variables, multiple options for treatment variables, donor variables, and options for trade-off and/or SOM analysis. Example outputs include results for the trade-off and/or SOM analysis. FIG. 13F is another example of GUI 1060 of trade-off UI 1006 demonstrating additional details of GUI inputs. Patient variables may generally be configure for a single patient. Treatment and donor variables may include multiple options. FIG. 13G is another example of GUI 1060 of trade-off UI 1006 demonstrating variations in inputs based on selections made. In embodiments, selection options may be reduced based on compatibility with selections already entered. FIG. 13H is another example of GUI 1060 of trade-off UI 1006 demonstrating an SOM mapping presentation of an outcomes view of results. FIG. 13I is another example of GUI 1060 of trade-off UI 1006 demonstrating a table presentation of an outcomes view of results. FIG. 13J is another example of GUI 1060 of trade-off UI 1006 demonstrating a predictor variables view of results with a predictor selected. In this example, a predictor of “conditioning intensity” is selected. FIG. 13K is another example of GUI 1060 of trade-off UI 1006 demonstrating a three-dimensional plot of risk scores presentation of an outcomes view of results. FIG. 13L is another example of GUI 1060 of trade-off UI 1006 demonstrating inputs for SOM mapping.

Shapley UI 1008 provides insights into the impact of various factors on the model's predictions. Shapley UI 1008 offers a view into the predictive model's logic, serving as a transparent window into the algorithm's considerations and influences. Shapley UI 1008 enables to the system to not only provide answers but also explain the rationale behind each answer or prediction provided, which is essential for trust and efficacy in clinical settings.

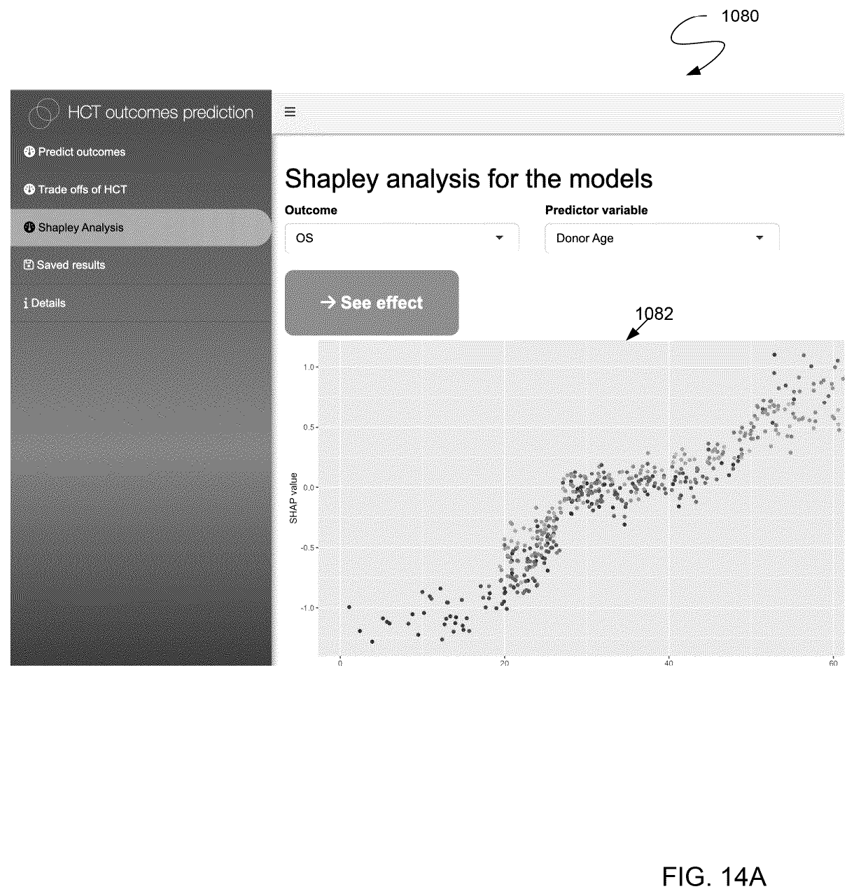

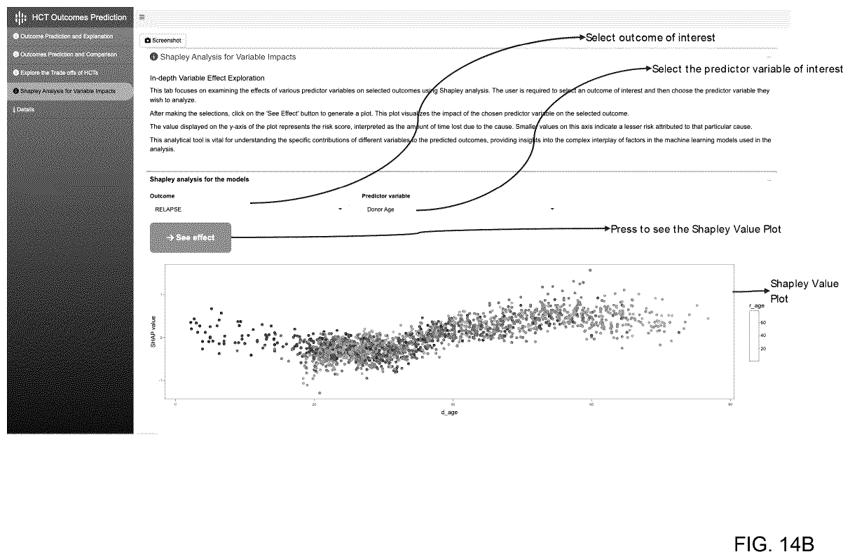

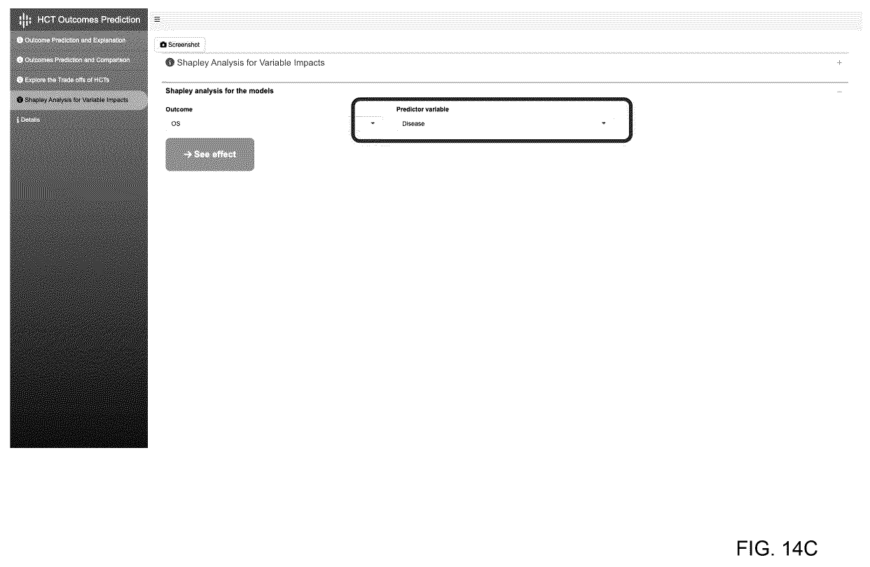

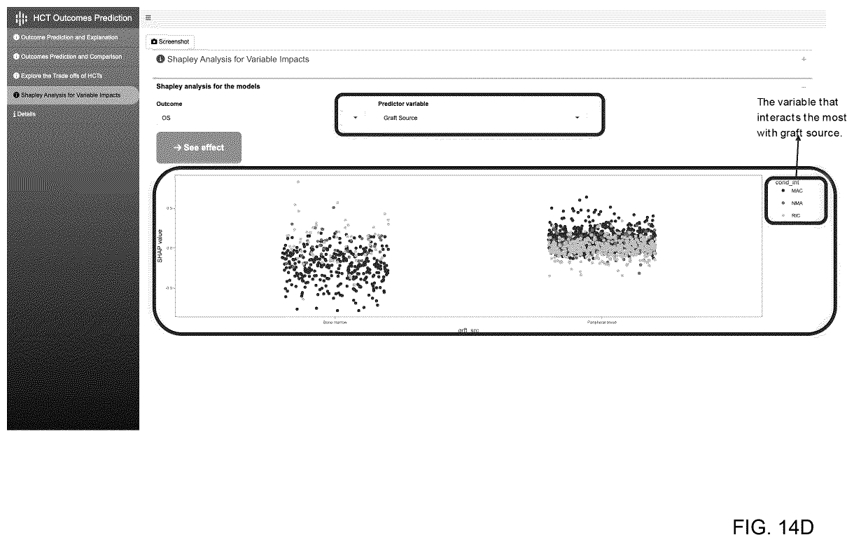

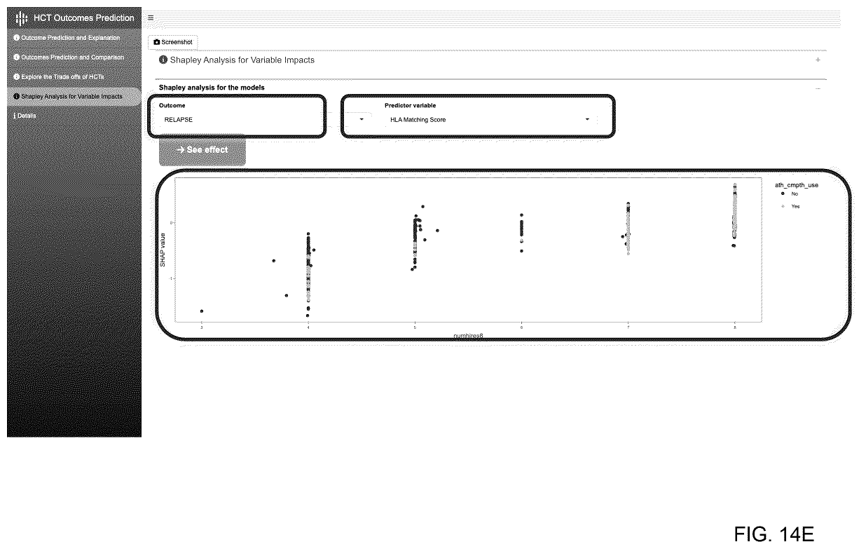

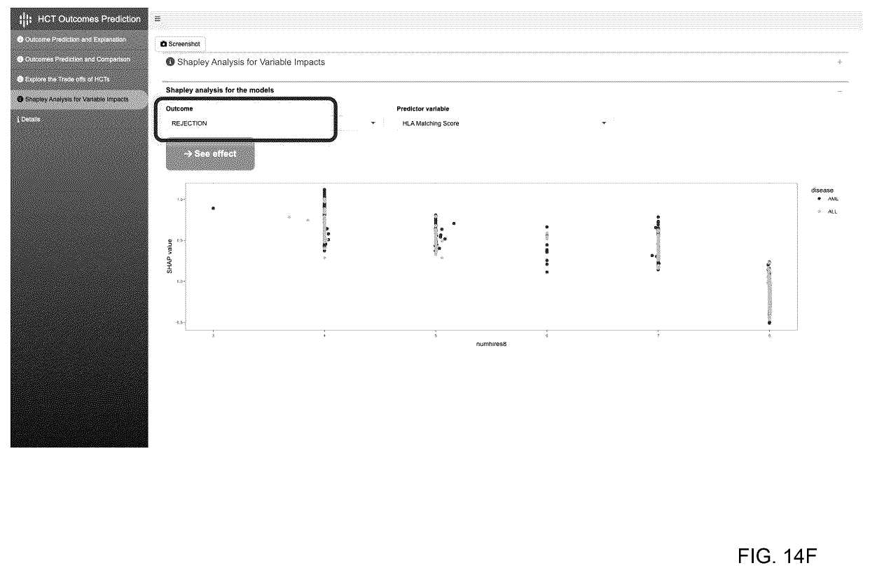

FIG. 14A is an example GUI 1080 depicting a Shapley UI 1008 of FIG. 11. Shapley plot 1082 presents an illustration of the underpinnings of the predictive model, which articulates the varying impacts of individual variables on the forecasted patient outcomes. After generating a prediction, clinicians can engage with the relevant model on a granular level through the Shapley UI 1008, exploring dependence plots that reveal the intricate balance of factors influencing survival, thereby enhancing the interpretability and transparency of the model's predictions.

FIGS. 14B-14F are further examples of GUI 1080 of FIG. 14A, depicting various example operations of Shapley UI 1008. FIG. 14B is another example of GUI 1080 of Shapley UI 1008 demonstrating inputs and outputs of the GUI 1080. Inputs include selection of an outcome and a predictor variable, and an operation button to operate the UI. Outputs include, for example, a Shapley value plot. FIG. 14C is another example of GUI 1080 of Shapley UI 1008 demonstrating further detail of the predictor variable selection. In this example, predictor variables may be selected from a dropdown menu. FIG. 14D is another example of GUI 1080 of Shapley UI 1008 demonstrating further detail of the Shapely value plot. FIG. 14E is another example of GUI 1080 of Shapley UI 1008 demonstrating another example plot with different inputs. FIG. 14F is another example of GUI 1080 of Shapley UI 1008 demonstrating output changes from the example of FIG. 14E with different inputs.

Saved Results UI 1010 Enables users to review and compare previously saved results. Saved Results UI 1010 functions as an archival vault, a feature that not only aids in immediate analytical comparisons but also serves the broader scope of longitudinal research, a feature indicative of the system's utility in progressive patient care and clinical study.

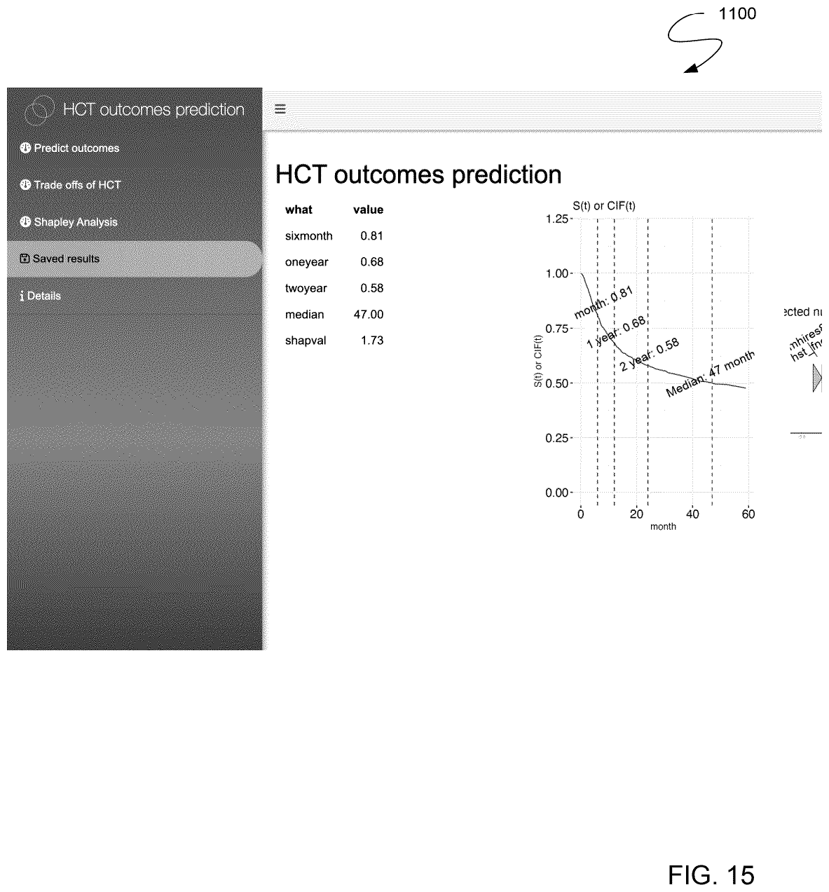

FIG. 15 is an example GUI 1100 depicting a saved results UI 1010 of FIG. 11. The saved results UI 1010 provides a strategic review platform where users can access, compare, and analyze outcomes from previously executed predictions.

Details UI 1012 offers detailed information and guidance on using the application. Details UI 1012 provides a compendium of the application's functionalities, providing users with the knowledge and guidance to effectively employ the application and its resources effectively. It is a resource that underscores the system's dedication to user empowerment and education in the complex field of HSCT.

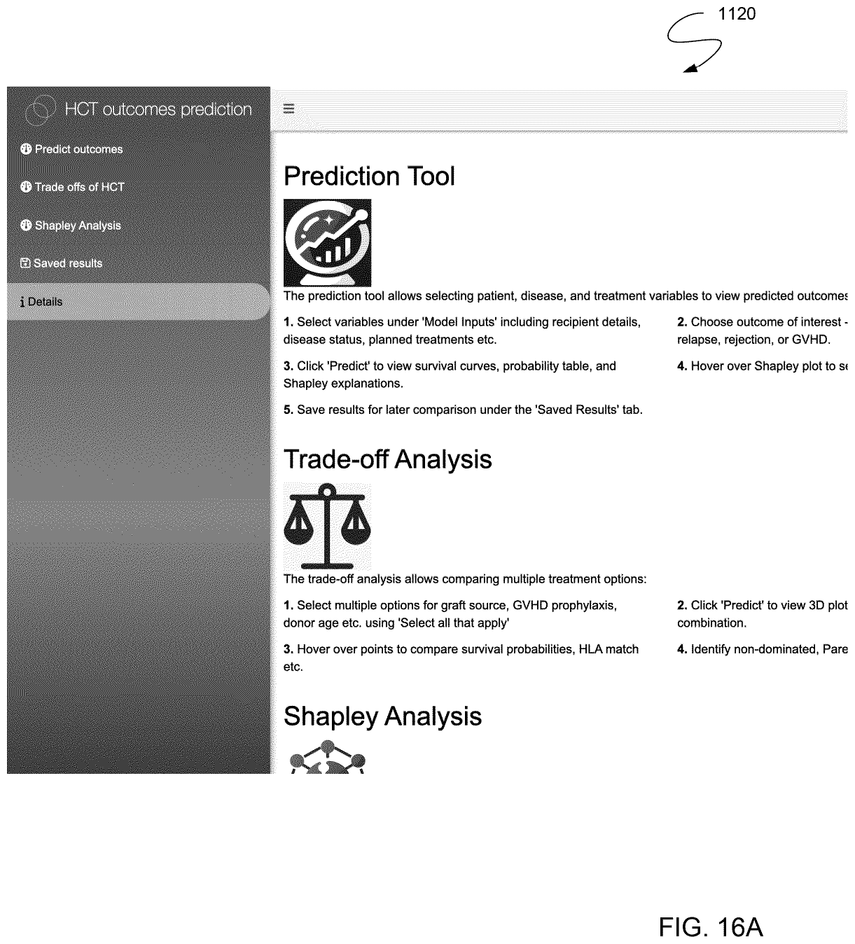

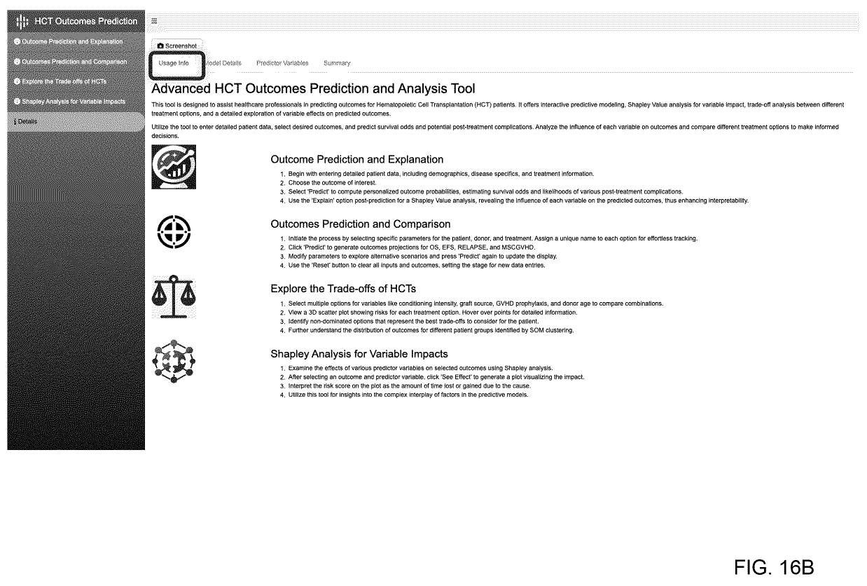

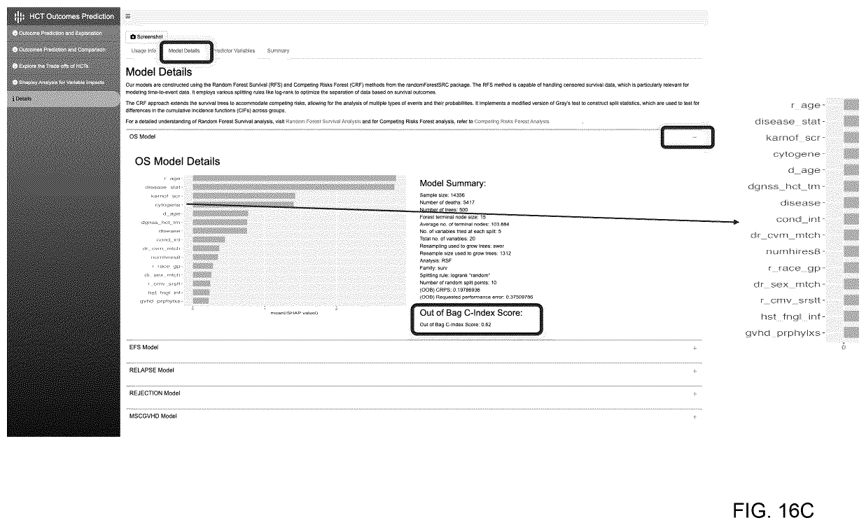

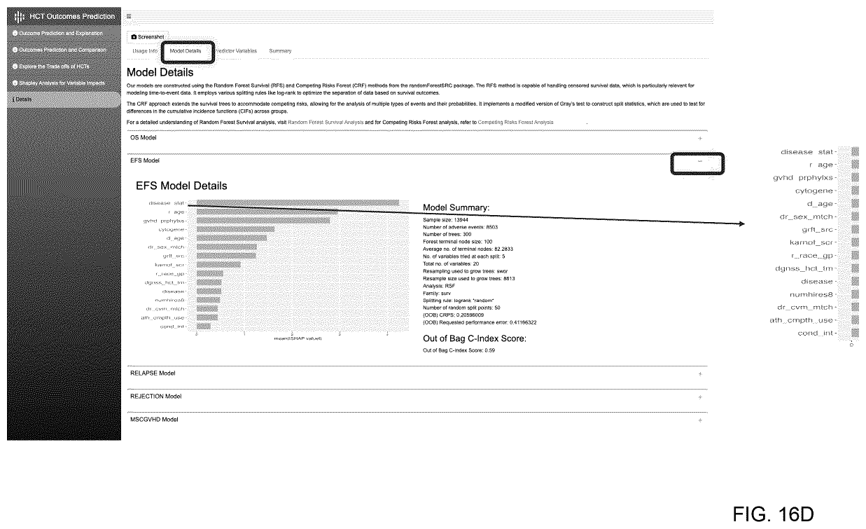

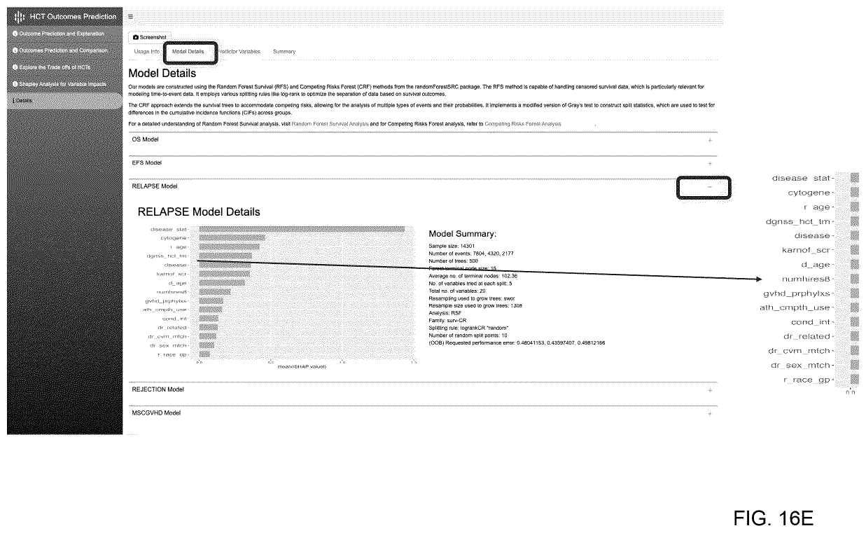

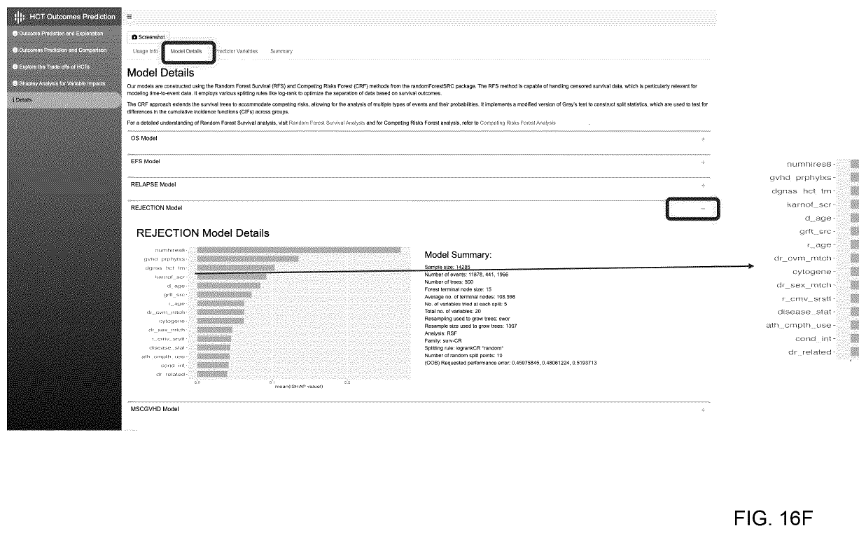

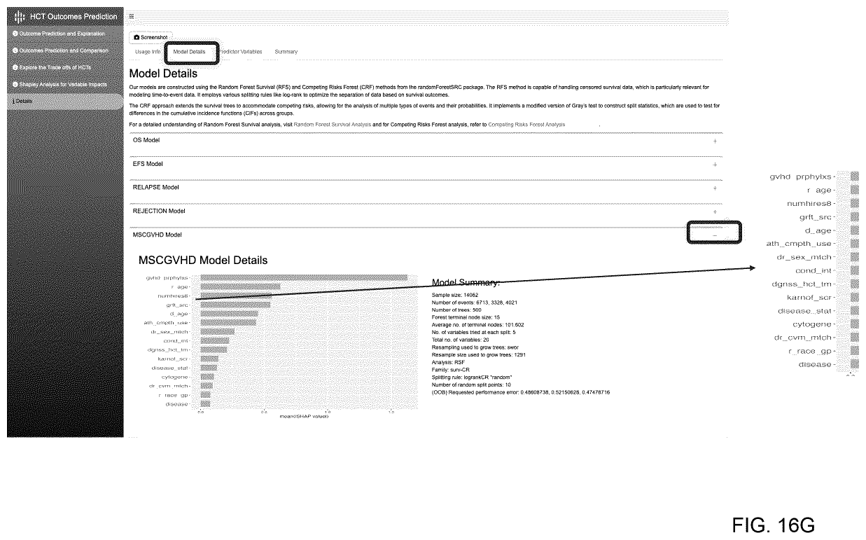

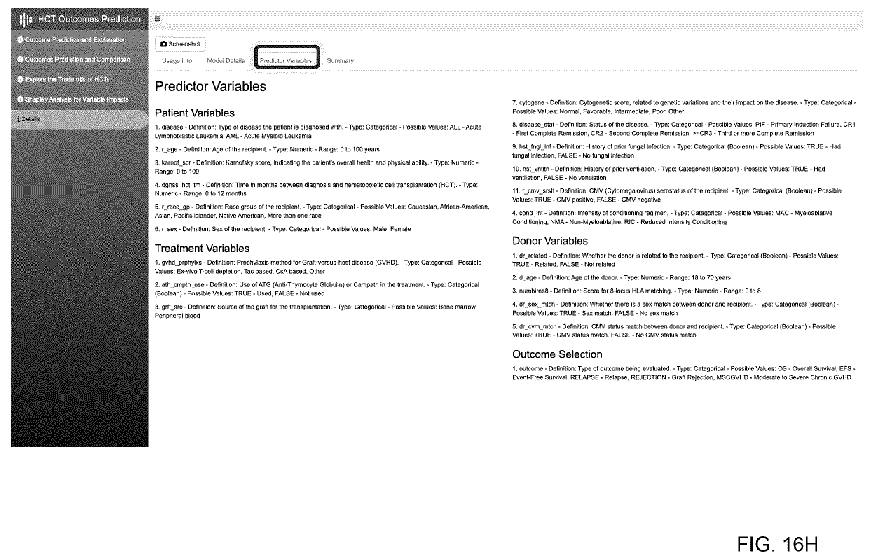

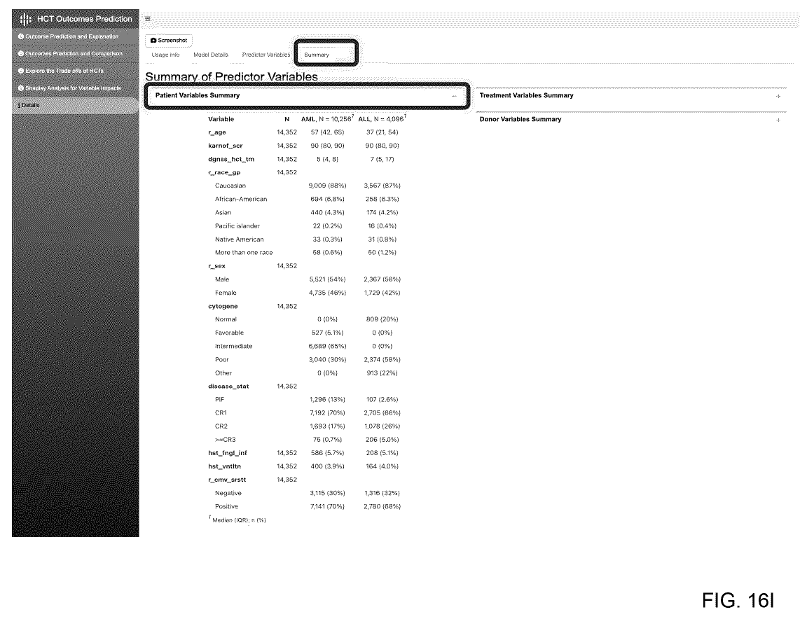

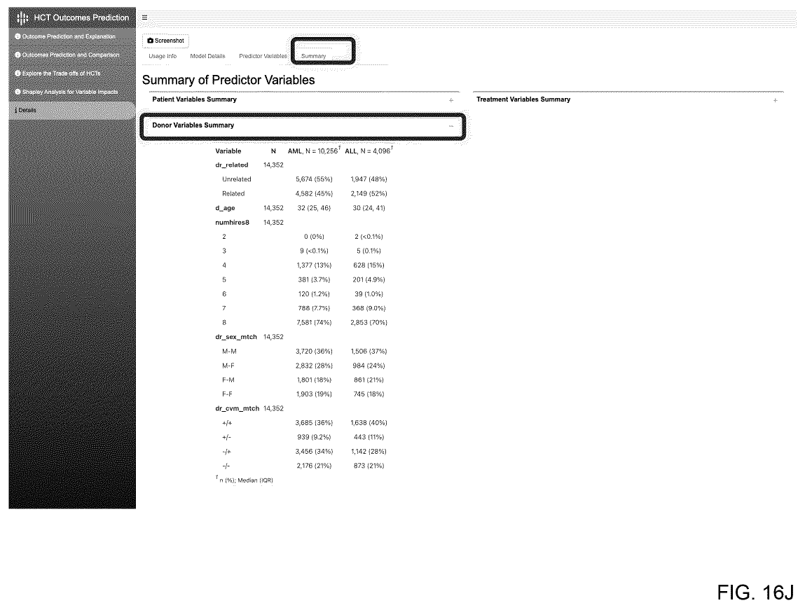

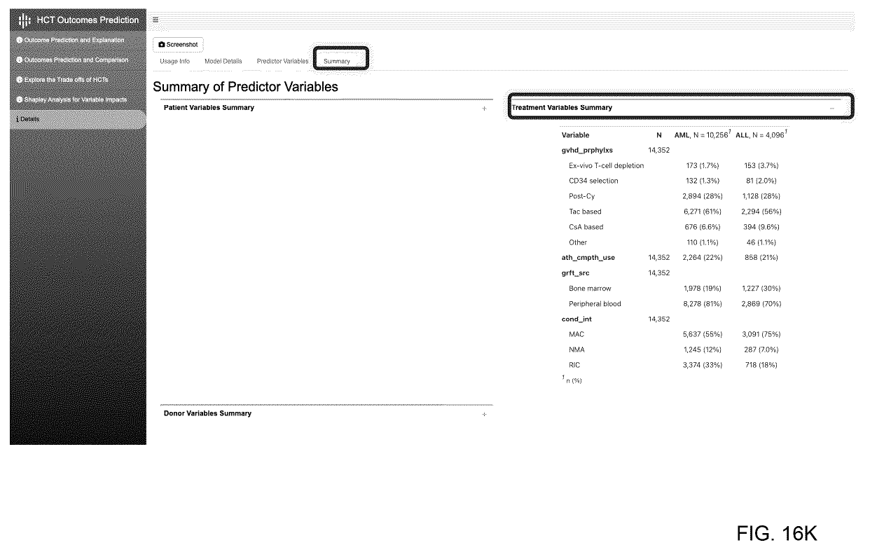

FIG. 16A is an example GUI 1120 depicting a details UI 1012 of FIG. 11. Details UI 1012 serves as an informative hub providing comprehensive guidance on navigating and utilizing the HSCT Explorer application.

FIG. 16B-16K are further examples of GUI 1120 of FIG. 16A, depicting various example operations of details UI 1012. FIG. 16B is another example of GUI 1120 of details UI 1012 demonstrating an usage information page. FIG. 16C is another example of GUI 1080 of Shapley UI 1008 demonstrating a model details view of an overall survival outcome model. FIG. 16D is another example of GUI 1080 of Shapley UI 1008 demonstrating the model details view of an event-free survival outcome model. FIG. 16E is another example of GUI 1080 of Shapley UI 1008 demonstrating the model details view of a relapse outcome model. FIG. 16F is another example of GUI 1080 of Shapley UI 1008 demonstrating the model details view of a rejection outcome model. FIG. 16G is another example of GUI 1080 of Shapley UI 1008 demonstrating the model details view of a graft-versus-host disease outcome model. FIG. 16H is another example of GUI 1080 of Shapley UI 1008 demonstrating a predictor variables view. In this example, various selectable variables are listed with definitions and associated values. FIG. 16I is another example of GUI 1080 of Shapley UI 1008 demonstrating a summary view of predictor variables. FIG. 16J is another example of GUI 1080 of Shapley UI 1008 demonstrating a summary view of donor variables. FIG. 16K is another example of GUI 1080 of Shapley UI 1008 demonstrating a summary view of treatment variables.

User inputs and selections in these tabs trigger backend processes within the server 1016. In embodiments, processing in the server 1016 is categorized under data handling and server response. These server-side processes manage data processing, response generation, and the delivery of results back to the user interface. The seamless interaction between the UI tabs 1002 and server 1016 processing ensures an intuitive and efficient user experience, making complex data analyses accessible and actionable for clinicians and researchers in the HSCT domain.

Referring to FIGS. 1-16 generally, it is noted that the diagnostic analysis server 12 of the present application implements a predictive analysis and decision support system in hematopoietic cell transplantation (HCT) which has a number of particularized features. In particular, the diagnostic analysis server 12 provides an artificial intelligence framework that utilizes a plurality of machine learning algorithms for predicting patient-specific risks associated with HCT as noted above. Such risks may include, but are not limited to, Overall Survival (OS) Event-Free Survival (EFS), relapse, rejection, and mile to severe chronic Graft-Versus-Host Disease (mscGHVD). The diagnostic analysis server 12 may employ a multi-objective optimization using Pareto optimality and non-dominated options for evaluating and presenting treatment paths and associated outcomes.

As further noted, the diagnostic analysis server 12 may present a variety of user interface features that improve ability to analyze such data. In particular, three-dimensional plots, self-organizing maps, and tabular representations are each used to explore and navigate treatment options within the complex, multidimensional data domain dictated by HCT. An interactive user interface enables input, processing, and visualization of patient data, model predictions, and comparative treatment analysis. Each of a variety of sets of analyses may be easily accessed via categorized user interface regions, for example separated into predictive and decision-support processes such as predictions, trade-offs, Shapley analysis, saved results, and details. Accordingly, detailed analytical outputs are easily accessed within the user interface, thereby enhancing the decision-making process.

While particular uses of the technology have been illustrated and discussed above, the disclosed technology can be used with a variety of data structures and processes in accordance with many examples of the technology. The above discussion is not meant to suggest that the disclosed technology is only suitable for implementation with the data structures shown and described above. For examples, while certain technologies described herein were primarily described in the context of queueing structures, technologies disclosed herein are applicable to data structures generally.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., operations, memory arrangements, etc.) described with respect to the figures herein are not intended to limit the technology to the particular aspects described. Accordingly, additional configurations can be used to practice the technology herein and/or some aspects described can be excluded without departing from the methods and systems disclosed herein.

Similarly, where operations of a process are disclosed, those operations are described for purposes of illustrating the present technology and are not intended to limit the disclosure to a particular sequence of operations. For example, the operations can be performed in differing order, two or more operations can be performed concurrently, additional operations can be performed, and disclosed operations can be excluded without departing from the present disclosure. Further, each operation can be accomplished via one or more sub-operations. The disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

What is claimed is:

1. A method of evaluating outcomes of a hematopoietic stem cell transplant (HSCT) and other cellular therapies for a patient, the method comprising:
receiving a set of patient characteristics;
defining one or more treatment options;
generating data based on the set of patient characteristics and one or more treatment options, including at least one actionable variable;
determining a risk score associated with one or more defined outcomes, based on the data;
evaluating an impact on the outcome associated with at least one actionable variable;
characterizing the impact of at least one actionable variable on the outcome over a range of associated values associated with at least one actionable variable to produce a patient set of treatment options based on one or more treatment options;
identifying a nondominated option from among the patient set of treatment options; and
generating a visualization of the nondominated option.

2. The method of claim 1, wherein the range of values is set according to the one or more treatment options.

3. The method of claim 1, further comprising training, for each outcome of the one or more outcomes, a machine learning model, to determine the risk score associated with the outcome.

4. The method of claim 3, wherein the machine learning model is a survival outcome model or a competing risks outcome model.

5. The method of claim 1, further comprising training for at least one outcome of the one or more outcomes, two or more machine learning models; and
training for the at least one outcome an ensemble machine learning model based on output generated using the two or more machine learning models.

6. The method of claim 1, wherein characterizing the impact of the at least one actionable variable on the outcome over a range of values comprises performing a Pareto optimization.

7. The method of claim 6, wherein identifying the nondominated option is based on the Pareto optimization.

8. The method of claim 1, further comprising generating a self-organizing map based on the patient dataset and the risk score.

9. The method of claim 8, wherein identifying the nondominated option is based on the self-organizing map.

10. The method of claim 1, wherein the impact is evaluated using one or more of variable importance scores, dependence analysis, and exponentiated coefficient estimates.

11. The method of claim 1, wherein the outcome is one or more of overall survival, effect free survival, rejection, relapse, or graft-versus-host disease.

12. A system for evaluating one or more outcomes of a hematopoietic stem cell transplant (HSCT) for a patient, the system comprising:
a processor;
a non-transitory memory in communication with the processor and storing instructions that, when executed, cause the processor to generate:
a risk assessor including one or more machine learning models, each of the one or more machine learning models associated with an outcome and configured to generate a risk score associated with the outcome based on a patient dataset, wherein the patient dataset is associated with a set of treatment options and includes at least one actionable variable; and
a trade-off analyzer including a multi-objective optimization framework, wherein the trade-off analyzer uses the multi-object optimization framework to:
characterize an impact on the risk score associated with the actionable variable across a range of values for the actionable variable; and
identify a non-dominated treatment option based on the set of treatment options, the range of values, and the impact.

13. The system of claim 12, wherein the multi-objective optimization framework comprises a Pareto optimization.

14. The system of claim 12, wherein the trade-off analyzer further comprises a self-organizing map.

15. The system of claim 12, further comprising a user interface, wherein the trade-off analyzer can receive, via the user interface, the range of values for the actionable variable.

16. The system of claim 12, wherein the actionable variable is associated with a HSCT donor characteristic.

17. The system of claim 12, wherein the patient dataset comprises a plurality of predictor variables.

18. The system of claim 17, wherein the risk assessor evaluates, for each predictor variable of the plurality of predictor variables, an impact value on the risk score.

19. The system of claim 18, wherein the outcome comprises a first outcome and a second outcome; and
wherein the risk assessor evaluates, for each predictor variable of the plurality of predictor variables, a first impact value associated with the first outcome and a second impact value associated with the second outcome.

20. The system of claim 19, wherein the risk assessor generates a visualization comparing the first impact value with the second impact value.
